SEQUENCE LISTING (142 sequences)

SEQ ID NO: 1 - protein, 121 aa, Artificial Sequence
  Artificial sequence from phage library screening
QLQLVESGGGTVQTGGSLRLACVVSGPTYSFYDMGWFRRAPGKEREFVAAVTASGDTTIYADSVKGRFTIARDNAKNTVYLQMNSLKSEDTGAYYCAAGEYSSTAATYYYWGQGTQVTVSS

SEQ ID NO: 2 - protein, 115 aa, Artificial Sequence
  Artificial sequence from phage library screening
QVQLVESGGGLVQPGGSLRLSCAASGSIFSVNEMGWYRQAPGKLRELVAAITSDGTTAYADSVKGRFTISRDNAKNTVYLQMNSLKPEDTAVYDCNTWPEARGDWGQGTQVTVSS

SEQ ID NO: 3 - protein, 116 aa, Artificial Sequence
  Artificial sequence from phage library screening
QLQLVESGGGLVQTGDSTKLSCTASGRSFSINDMGWYRQAPGKLRELVAGITSGGSTNYADSVKGRFTISRDNAKNTVYLQMNSLKPEDTAVYYCNVLGWPDFGSWGQGTQVTVSS

SEQ ID NO: 4 - protein, 114 aa, Artificial Sequence
  Artificial sequence from phage library screening
QVQLVESGGGLVQPGGSLRLSCAASGSIFKINAMGWYRQAPGKLRELVAGITSGGSTNYADSVKGRFTISRDNAKNTLYLQMNSLKPDDTAVYYCNQLGFPQYWGQGTQVTVSS

SEQ ID NO: 5 - protein, 130 aa, Artificial Sequence
  Artificial sequence from phage library screening
EVQVVESGGGLVQAGGSLTLSCAASGRPFSTFPTSMGWFRQAPGKEREFVAAISESGLNTYYRDSVKGRFGISRDNAKNTVYLQMNTLTAEDTAVYFCAAAPGYMTTDSQYYLGNKYQYWGQGTQVTVSS

SEQ ID NO: 6 - protein, 124 aa, Artificial Sequence
  Artificial sequence from phage library screening
QVQLVESGGGLVQPGGSLRLSCAASRAIFPEYSIAWFRQAPGKEREGVALVSRIGDRVDYADSVKGRFTISRDNAKNTVYLQMNSLKPEDTAVYHCAAGQLSSSTYYGDEYVYWGQGTQVTVSS

SEQ ID NO: 7 - protein, 125 aa, Artificial Sequence
  Artificial sequence from phage library screening
EVQVVESGGGLVQAGGSLRLSCAASGRTFSNYNVGWFRQAPGKEREFLATISESGGSTYYADSVKGRFTISRDNGINMVYLQMSSLKPEDTAVYYCAESRIFSFSYADGRGYAYWGQGTQVTVFS

SEQ ID NO: 8 - protein, 126 aa, Artificial Sequence
  Artificial sequence from phage library screening
QVQLVESGGGLVQTGDSLRLSCAASGRTYDNYAMGWFRQAPGKEREFVAAFSQNGGRTDYADSVKGRFAISDDRSTVHLDMNSLLPEDTAVYYCAARPPAYFSNAVYSKVDEYRYWGQGTQVTVSS

SEQ ID NO: 9 - protein, 124 aa, Artificial Sequence
  Artificial sequence from phage library screening
QVQLVESGGGSVQAGGSLRLSCAASGRTFSDYVMGWFRQAPGKEREFVGTITRIGGMPYYADFVKGRFTISRDYAKNAGYLQLNNLKPEDTALYYCALDLRLRLGVTPREYDYWGQGTQVTVSS

SEQ ID NO: 10 - protein, 115 aa, Artificial Sequence
  Artificial sequence from phage library screening
QVQLVESGGGLVQPGGSLTLSCAVSGITFSRSVMGWFRQAPGKLRELVAGITSDDNTDYADSVKGRFTISRDNAKNTVYLQMNSLKPEDTAVYYCNILPDSRLGWGQGTQVTVSS

SEQ ID NO: 11 - protein, 115 aa, Artificial Sequence
  Artificial sequence from phage library screening
EVQLVESGGGLVQPGESLRLSCVASGGDFRFKSMGWYRQAPGKLRELVAGITTGGVTDYADSVKGRFTISRDNANKAVWLQMNRLKPDDTAVYDCNTLGAAAYDWGQGTQVTVSS

SEQ ID NO: 12 - protein, 114 aa, Artificial Sequence
  Artificial sequence from phage library screening
EVQLVESGGDLVQPGGSLRLSCAAFGSSFSIDMMGWYRQVPGKQRELVAGITKGGFADYADSVKGRFTISMDAAKNTVYLQMNSLKPEDTAVYYCNTLPTHSDWGQGTQVTVSS

SEQ ID NO: 13 - protein, 116 aa, Artificial Sequence
  Artificial sequence from phage library screening
EVQLVESGGGLVQPGGSLRLSCAASGGIFGSTHMGWYRQAPGKLREMVAGITSTGNHRNVADSVKGRFTISKDNAKNTLYLQMNSLKPEDTAVYYCNSFPDQVFNWGQGTQVTVSS

SEQ ID NO: 14 - protein, 115 aa, Artificial Sequence
  Artificial sequence from phage library screening
QLQLVESGGGLVQPGGSLRLSCAVSASIFSVNEMGWYRQAPGKLRELVAAITSDGTTAYADSVKGRFTISRDNAKNTVYLQMNSLKPEDTAVYDCNTWPQARGDWGQGTQVTVSS

SEQ ID NO: 15 - protein, 121 aa, Artificial Sequence
  Artificial sequence from phage library screening
EVQLVESGGGSVQTGGSLRLSCAASGPTFNFYGMAWFRQAPGKEREFVASIIPGSGDTSYASSVRGRFTISEDNARNNAYLQMNTLKPEDTAVYYCAAGTGGIYSSDYPYWGQGTQVTVSS

SEQ ID NO: 16 - protein, 114 aa, Artificial Sequence
  Artificial sequence from phage library screening
EVQLVESGGGLVQAGGSLRLSCAASGISLITYNMGWYRQAPGKLRELVAGITTGGITDYTDSVKGRFTISRDNAKNTLYLQMNSLKPEDTAVYYCDTLGMANYWGQGTQVTVSS

SEQ ID NO: 17 - protein, 116 aa, Artificial Sequence
  Artificial sequence from phage library screening
QLQLVESGGGLVQPGGSLRLSCAASGSIDSINAMGWYRQAPGKLRELVAGITRGGDVNYADSVKGRFTISRVDARNTVTVYLQMNSLKPEDTAVYYCNNLGFPSDWAQGTQVTVSS

SEQ ID NO: 18 - protein, 114 aa, Artificial Sequence
  Artificial sequence from phage library screening
EVQLVESGGGLVQPGGSLRLSCAAAGIRFSIANMGWYRQAPGKLHEMVAGITSDGFTNYADSVKGRFTISRDNAKNTVYLQMNNLKPEDTAVYYCDSFPTRHYWGQGTQVTVSS

SEQ ID NO: 19 - protein, 114 aa, Artificial Sequence
  Artificial sequence from phage library screening
EVQVVESGGDLVQPGGSLRLSCAAFGSSFSIDMMGWYRQVPGKQRELVAGITKGGFADYADSVKGRFTISMDAAKNTVYLQMNSLKPEDTAVYYCNTLPTHSDWGQGTQVTVSS

SEQ ID NO: 20 - protein, 116 aa, Artificial Sequence
  Artificial sequence from phage library screening
EVQLVESGGGLVQPGGSLRLSCAASGSAFSIEPWGWYRQAPGQLRELVAGITSDDKTDYADSVKDRFTISRDNAKNTVFLQMNSLKPEDTAVYYCNVLGWPRDDYWGQGTQVTVSS

SEQ ID NO: 21 - protein, 123 aa, Artificial Sequence
  Artificial sequence from phage library screening
EVQLVESGGGLVQPGGSLRLSCAASGSTYAFYNMGWFRQRPGKEREFAAAILWGRSGVYTTYADSVKGRFTISRDNAQNTVYLQMNSLKPEDTAVYYCAAGLGTMRANEYDYWGQGTQVTVSS

SEQ ID NO: 22 - protein, 117 aa, Artificial Sequence
  Artificial sequence from phage library screening
QVQLVESGGGLVQPGGSLRLSCAASGSIFSANVMGWYRQAPGKLRELVAGMHSGGSANYADSVKGRFTISKDNAKNTMYLQMNSLKPEDTAVYYCNFGLGFDGHDYWGQGTQVTVSA

SEQ ID NO: 23 - protein, 114 aa, Artificial Sequence
  Artificial sequence from phage library screening
EVQLVESGGGLVQPGGSLRLSCAASGSIFSPNGIGWYRQAPGKLRELVAGITGNGLTDYADSVKGRFTISRDSAKNTVYLQMNSLKTEDTAVYYCNLLLNHAEWGQGTQVTVSS

SEQ ID NO: 24 - DNA, 387 bp, Artificial Sequence
  synthetic polynucleotide
cagttgcagctcgtggagtctgggggaggaacggtacaaactgggggctctctgagactcgcctgtgtagtctctggaccaacctacagtttctatgacatgggctggttccgccgggctccagggaaggagcgtgaatttgtggcagctgttacggcgagcggtgatactacaatctatgcagactccgtgaagggccgattcaccatcgccagagacaacgccaagaacacggtgtatctgcaaatgaacagcctgaaatctgaggacacgggcgcgtattactgtgcagcgggagagtatagtagtaccgctgccacctattactactggggccaggggacccaggtcaccgtctcctcagcgcaccacagcgaagaccccagc

SEQ ID NO: 25 - DNA, 369 bp, Artificial Sequence
  synthetic polynucleotide
caggtgcagctcgtggagtcagggggaggcttggtgcagcctggggggtctctgagactctcctgtgcagcctctggaagcatcttcagtgtcaatgagatgggctggtaccgccaggctccagggaagctgcgcgagttggtcgccgctattactagcgatggtaccacggcatatgcagactccgtgaagggccgattcaccatctccagagataacgccaagaacacggtgtatctgcaaatgaacagcctgaaacctgaggacacggccgtctatgactgtaatacttggccagaggcacgtggcgactggggccaggggacccaggtcaccgtttcctcagcgcaccacagcgaagaccccagc

SEQ ID NO: 26 - DNA, 375 bp, Artificial Sequence
  synthetic polynucleotide
cagttgcagctcgtggagtccgggggaggattggtacagactggggactctacgaaactctcctgtacagcctctggacgcagcttcagtatcaatgacatgggctggtaccgccaggctccagggaagctgcgcgagttggtcgccggtattactagtggtggtagcacaaactatgcagactccgtgaagggccgattcaccatctccagagacaacgccaagaacacggtgtatctgcaaatgaacagcctgaaacctgaggacacggccgtctattactgtaatgttttgggctggcctgattttggttcctggggccaggggacccaggtcaccgtctcctcggaacccaagacaccaaaaccacaacca

SEQ ID NO: 27 - DNA, 369 bp, Artificial Sequence
  synthetic polynucleotide
caggtgcagctcgtggagtcggggggaggcttggtgcagcctggggggtctctgagactctcctgtgcagcctctggaagcatcttcaagatcaatgccatgggctggtaccgccaggctccagggaagctgcgcgagttggtcgcaggtattactagtggtggtagtacaaactatgcagactccgtgaagggccgattcaccatctccagagacaacgccaagaacacgctgtatctgcaaatgaacagcctgaaacctgacgacacggccgtctattactgcaatcaactgggatttccacagtactggggccaggggacccaggtcaccgtctcctcagaacccaagacaccaaaaccacaacca

SEQ ID NO: 28 - DNA, 417 bp, Artificial Sequence
  synthetic polynucleotide
gaggtgcaggtggtggagtccggtggaggattggtgcaggctggcggctctctgaccctctcctgtgcagcctctggacgccccttcagtacatttccaacatccatgggctggttccgccaggctccagggaaggagcgtgagtttgtagcagcaattagcgagagtgggcttaatacatactatcgagactccgtgaagggccgattcggcatctccagagacaacgccaagaacacggtgtatctgcagatgaacactttgacagctgaagacacggccgtttatttctgtgcagcggcgcccggttacatgaccactgatagccagtactacttgggcaacaagtatcagtactggggccaggggacccaggtcaccgtctcctcagaacccaagacaccaaaaccacaacca

SEQ ID NO: 29 - DNA, 399 bp, Artificial Sequence
  synthetic polynucleotide
caggtgcagctcgtggagtccgggggaggcttagtgcagcctggggggtctctgagactctcctgtgctgcctctagggccatctttcctgaatatagtatagcctggttccgccaggccccagggaaggagcgtgagggggtcgcattggttagtaggattggtgatagggtagactatgcagactccgtgaagggccggttcaccatctccagagataacgccaagaatacggtgtatctgcaaatgaacagcctgaaacctgaggacacagccgtttatcattgtgcagcaggtcaactgtcttcgtctacttattatggggatgagtatgtctactggggccaggggacccaggtcaccgtctcctcagaacccaagacaccaaaaccacaacca

SEQ ID NO: 30 - DNA, 402 bp, Artificial Sequence
  synthetic polynucleotide
gaggtgcaggtggtggagtctggtggaggattggtgcaggctgggggctctctgagactctcctgtgcagcctctggacgcaccttcagtaactacaacgtgggctggttccgccaggctccagggaaggagcgtgagttcctagcaactattagcgagagtggtggcagtacatactatgcagactccgtgaagggccgattcaccatctccagagacaacggcatcaacatggtgtatctgcagatgagcagcctgaaaccagaggacacggccgtttattactgtgctgaatcacgaatcttcagttttagttacgccgacggaagagggtatgcctactggggccaggggacccaggtcaccgtcttttccgaacccaagacaccaaaaccacaacca

SEQ ID NO: 31 - DNA, 405 bp, Artificial Sequence
  synthetic polynucleotide
caggtgcagctcgtggagtccgggggaggattggtgcagactggggactctctgagactctcctgtgcagcctctggacgcacctacgataactatgccatgggctggttccgccaggctccagggaaggagcgtgagtttgtagcagcctttagccagaatggtggtagaacagactatgcggactccgtgaagggccgcttcgccatctccgacgacaggagtacggtgcatctcgatatgaacagcctgttacctgaggacacggccgtttattactgtgcagcgagacccccggcatactttagtaatgctgtctactcaaaagtcgatgagtatcgttactggggccaggggacccaggtcaccgtgtcctcagaacccaagacaccaaaaccacaacca

SEQ ID NO: 32 - DNA, 399 bp, Artificial Sequence
  synthetic polynucleotide
caggtgcagctcgtggagtcgggtggaggatcggtgcaggctgggggctcactgagactctcctgtgcagcctctggacgcaccttcagtgactacgtcatgggctggttccgccaggctccagggaaggagcgtgagtttgtgggaactattacccgcattggtggtatgccatactatgcagacttcgtgaagggccgattcaccatctccagagactacgccaagaacgcgggatatctgcaactgaacaacctgaaacctgaggacacggccctttattactgtgcactagacctccgactacggcttggggtgaccccgcgggagtatgactactggggccaggggacccaggtcaccgtctcctcagaacccaagacaccaaaaccacaacca

SEQ ID NO: 33 - DNA, 372 bp, Artificial Sequence
  synthetic polynucleotide
caggtgcagctcgtggagtccgggggaggcttggtgcagcctggggggtctctgacgctctcctgtgcagtctctggaatcaccttcagtaggtcagttatgggctggttccgccaggctccagggaagctgcgtgagttggtcgcaggtattacgagtgatgataacactgactatgcagactccgtgaagggccgattcaccatctccagagacaacgccaagaatacggtgtatctgcaaatgaacagcctgaaacctgaggacacggccgtctattactgtaatatattgccggacagccggctggggtggggccaggggactcaggtcaccgtctcctcagaacccaagacaccaaaaccacaacca

SEQ ID NO: 34 - DNA, 372 bp, Artificial Sequence
  synthetic polynucleotide
gaggtgcagctggtggagtccgggggaggcttggtgcagcctggggaatctctgagactctcctgtgtagcctctggaggcgactttcgtttcaagtccatgggctggtaccgccaggctccagggaagctgcgcgagttggtcgcaggaattaccacgggtggtgttacggactatgcagactccgtcaagggccgattcaccatctccagagacaacgccaacaaagcggtgtggctgcaaatgaacagattgaaacctgacgacacggccgtctatgactgtaatactcttggcgccgctgcttatgactggggccaggggacccaggtcaccgtctcctccgaacccaagacaccaaaaccacaacca

SEQ ID NO: 35 - DNA, 369 bp, Artificial Sequence
  synthetic polynucleotide
gaggtgcagctggtggagtccgggggagacttggtgcagcctggggggtctctgagactctcctgtgcagctttcggaagcagcttcagtatcgatatgatgggctggtaccgccaggttccagggaagcagcgcgagttggtcgcaggcattactaaaggtgggttcgccgactatgcagactccgtgaagggccgattcaccatctccatggacgccgccaagaacacggtgtatctgcaaatgaacagcctgaaacctgaggacacggccgtctattactgtaatacgctccctacgcactctgactggggccaggggacccaggtcaccgtctcctcagaacccaagacaccaaaaccacaacca

SEQ ID NO: 36 - DNA, 375 bp, Artificial Sequence
  synthetic polynucleotide
gaggtgcagctggtggagtccgggggaggcttggtgcagcctggggggtctctgagactctcctgtgcagcctctggaggcatcttcggtagtactcacatgggctggtaccgccaggctccagggaagttgcgcgaaatggtcgcgggaattactagcactggtaaccacagaaacgtcgcagactccgtgaagggccgattcaccatctccaaggacaacgccaagaacacgctgtatctgcaaatgaacagcctgaaacctgaggacacggccgtctattactgtaattcctttcccgaccaagtatttaactggggccaagggacccaggtcaccgtctcctcggaacccaagacaccaaaaccacaacca

SEQ ID NO: 37 - DNA, 372 bp, Artificial Sequence
  synthetic polynucleotide
cagttgcagctcgtggagtccgggggaggcttggtgcagcctggggggtctctgagactctcctgtgcagtctctgcaagcatcttcagtgtcaatgagatgggctggtaccgccaggctccagggaagctgcgcgagttggtcgccgctattactagcgatggtaccacggcatatgcagactccgtgaagggccgattcaccatctccagagataacgccaagaacacggtgtatctgcaaatgaacagcctgaaacctgaggacacggccgtctatgactgtaatacttggccacaggcacgtggcgactggggccaggggacccaggtcaccgtttcctcagaacccaagacaccaaaaccacaacca

SEQ ID NO: 38 - DNA, 390 bp, Artificial Sequence
  synthetic polynucleotide
gaggtgcagctggtggagtcagggggaggatcggtgcagactgggggctctctgagactctcctgtgcagcctctggacccaccttcaatttctatggcatggcctggttccgccaggctccaggcaaggagcgagaatttgtagcaagtattattccgggtagtggtgacacaagctatgcatcctccgtgaggggccgattcaccatctccgaagacaacgcccggaacaatgcatatctgcaaatgaacaccctgaaacctgaggacacggcggtttattactgtgcagcgggaactggtggtatctattcaagtgactatccttactggggccaggggacccaggtcaccgtctcctcagaacccaagacaccaaaaccacaacca

SEQ ID NO: 39 - DNA, 369 bp, Artificial Sequence
  synthetic polynucleotide
gaggtgcagctggtggagtcggggggaggcttggtgcaggctggggggtctctgagactctcctgtgcagcctctggaatcagcctcattacctataacatgggctggtaccgccaggctccagggaagctgcgtgagttggtcgcaggtattactactggtggtatcaccgattatacagactccgtgaagggccgattcaccatctccagagacaacgccaagaacacgctgtatctgcaaatgaacagcctgaaacctgaggacacggccgtctattactgtgacaccctagggatggcgaactactggggccaggggacccaggtcaccgtctcctcagaacccaagacaccaaaaccacaacca

SEQ ID NO: 40 - DNA, 375 bp, Artificial Sequence
  synthetic polynucleotide
cagttgcagctcgtggagtcggggggaggcttggtgcagcctggggggtctctgagactctcctgtgcagcctctggaagcatcgacagtatcaatgccatgggctggtaccgccaggctccagggaagctgcgcgagttggtcgcgggaattactagaggtggtgacgtaaattatgcagactccgtgaagggccgattcaccatctccagagtcgacgccaggaacacggtgacggtgtatctgcaaatgaacagcctgaaacctgaggacacggccgtctattactgtaataatttggggtttccttccgactgggcccaggggacccaagtcaccgtctcctcagaacccaagacatcaaaaccacaacca

SEQ ID NO: 41 - DNA, 369 bp, Artificial Sequence
  synthetic polynucleotide
gaggtgcagctggtggagtcaggtggaggcttggtgcagcctggggggtctctgagactctcctgtgcagccgctggaatcagattcagtatcgctaacatgggctggtaccgccaggctccagggaagctgcacgaaatggtcgcgggtattactagtgatggtttcacaaactatgcagactccgtgaagggccgattcaccatctccagagacaacgccaagaacacggtctatctacaaatgaacaacctgaaacccgaggacacggccgtctattactgcgattcgttccctacccgacactactggggtcaggggacccaggtcaccgtctcctcagaacccaagacaccaaaaccacaacca

SEQ ID NO: 42 - DNA, 369 bp, Artificial Sequence
  synthetic polynucleotide
gaggtgcaggtggtggagtcggggggagacttggtgcagcctggggggtctctgagactctcctgtgcagctttcggaagcagcttcagtatcgatatgatgggctggtaccgccaggttccagggaagcagcgcgagttggtcgcaggcattactaaaggtgggttcgccgactatgcagactccgtgaagggccgattcaccatctccatggacgccgccaagaacacggtgtatctgcaaatgaacagcctgaaacctgaggacacggccgtctattactgtaatacgctccctacgcactctgactggggccaggggacccaggtcaccgtctcctcagaacccaagacaccaaaaccacaacca

SEQ ID NO: 43 - DNA, 375 bp, Artificial Sequence
  synthetic polynucleotide
gaggtgcagctggtggagtcggggggaggcttggtgcagcctggggggtctctgagactctcctgtgcagcctctggaagtgccttcagtatcgaaccctggggctggtaccgccaggctccagggcaactgcgcgagttggtcgcaggtattacttcagatgataaaacagactatgcagactccgtgaaggaccgattcaccatctccagagacaatgccaagaacacggtgtttctgcaaatgaacagcctgaaacctgaggacacggccgtctattactgtaatgtactgggctggccgagagatgactactggggccaggggacccaggtcaccgtctcctcagaacccaagacaccaaaaccacaacca

SEQ ID NO: 44 - DNA, 396 bp, Artificial Sequence
  synthetic polynucleotide
gaggtgcagctggtggagtcagggggaggcttggtgcagccgggggggtctctgaggctctcctgtgcagcctctggaagtacctacgctttctataacatgggctggttccgccagcgtcctgggaaggagcgtgagtttgcagcagctatattatggggtcgcagtggggtctacacaacgtatgcagactccgtgaagggccgattcaccatctctagagacaacgcccagaacacggtgtatctgcaaatgaacagcctgaaacctgaggacacggccgtttattactgtgcagcaggcctcgggactatgcgggcaaatgaatatgactactggggccaggggacccaggtcaccgtctcctcagaacccaagacaccaaaaccacaacca

SEQ ID NO: 45 - DNA, 378 bp, Artificial Sequence
  synthetic polynucleotide
caggtgcagctcgtggagtcggggggaggcttggtgcagcctggggggtctctgagactctcctgtgcagcctctggaagcatcttcagtgccaatgtcatgggctggtaccgccaggctccagggaagctgcgcgagttggtcgcaggtatgcatagtggtggtagcgcaaactatgcagactccgtgaagggccgattcaccatctccaaagacaacgccaagaacacgatgtatctgcaaatgaacagcctgaaacctgaggacacggccgtctattactgtaatttcgggttggggtttgatgggcatgactactggggccaggggacccaggtcaccgtctccgcagaacccaagacaccaaaaccacaacca

SEQ ID NO: 46 - DNA, 369 bp, Artificial Sequence
  synthetic polynucleotide
gaggtgcagctggtggagtctgggggaggcttggtgcaacctggggggtctctgagactctcctgtgcagcctctggaagcatcttcagccccaatggcataggctggtaccgccaggctccggggaagttgcgcgaattggtcgcaggtattacaggcaacgggctcactgactatgcagactccgtgaagggccgattcaccatctccagagacagcgccaagaacacggtgtatctgcaaatgaacagcctgaaaaccgaagacacggccgtctattactgtaacctactattgaaccacgcggagtggggccaggggacccaggtcaccgtctcctcagaacccaagacaccaaaaccacaacca

SEQ ID NO: 47 - protein, 5 aa, Artificial Sequence
  synthetic peptide
FYDMG

SEQ ID NO: 48 - protein, 5 aa, Artificial Sequence
  synthetic peptide
VNEMG

SEQ ID NO: 49 - protein, 5 aa, Artificial Sequence
  synthetic peptide
INDMG

SEQ ID NO: 50 - protein, 5 aa, Artificial Sequence
  synthetic peptide
INAMG

SEQ ID NO: 51 - protein, 7 aa, Artificial Sequence
  synthetic peptide
TFPTSMG

SEQ ID NO: 52 - protein, 5 aa, Artificial Sequence
  synthetic peptide
EYSIA

SEQ ID NO: 53 - protein, 5 aa, Artificial Sequence
  synthetic peptide
NYNVG

SEQ ID NO: 54 - protein, 5 aa, Artificial Sequence
  synthetic peptide
NYAMG

SEQ ID NO: 55 - protein, 5 aa, Artificial Sequence
  synthetic peptide
DYVMG

SEQ ID NO: 56 - protein, 5 aa, Artificial Sequence
  synthetic peptide
RSVMG

SEQ ID NO: 57 - protein, 5 aa, Artificial Sequence
  synthetic peptide
FKSMG

SEQ ID NO: 58 - protein, 5 aa, Artificial Sequence
  synthetic peptide
IDMMG

SEQ ID NO: 59 - protein, 5 aa, Artificial Sequence
  synthetic peptide
STHMG

SEQ ID NO: 60 - protein, 5 aa, Artificial Sequence
  synthetic peptide
VNEMG

SEQ ID NO: 61 - protein, 5 aa, Artificial Sequence
  synthetic peptide
FYGMA

SEQ ID NO: 62 - protein, 5 aa, Artificial Sequence
  synthetic peptide
TYNMG

SEQ ID NO: 63 - protein, 5 aa, Artificial Sequence
  synthetic peptide
INAMG

SEQ ID NO: 64 - protein, 5 aa, Artificial Sequence
  synthetic peptide
IANMG

SEQ ID NO: 65 - protein, 5 aa, Artificial Sequence
  synthetic peptide
IDMMG

SEQ ID NO: 66 - protein, 5 aa, Artificial Sequence
  synthetic peptide
IEPWG

SEQ ID NO: 67 - protein, 5 aa, Artificial Sequence
  synthetic peptide
FYNMG

SEQ ID NO: 68 - protein, 5 aa, Artificial Sequence
  synthetic peptide
ANVMG

SEQ ID NO: 69 - protein, 5 aa, Artificial Sequence
  synthetic peptide
PNGIG

SEQ ID NO: 70 - protein, 17 aa, Artificial Sequence
  synthetic peptide
AVTASGDTTIYADSVKG

SEQ ID NO: 71 - protein, 16 aa, Artificial Sequence
  synthetic peptide
AITSDGTTAYADSVKG

SEQ ID NO: 72 - protein, 16 aa, Artificial Sequence
  synthetic peptide
GITSGGSTNYADSVKG

SEQ ID NO: 73 - protein, 16 aa, Artificial Sequence
  synthetic peptide
GITSGGSTNYADSVKG

SEQ ID NO: 74 - protein, 17 aa, Artificial Sequence
  synthetic peptide
AISESGLNTYYRDSVKG

SEQ ID NO: 75 - protein, 17 aa, Artificial Sequence
  synthetic peptide
LVSRIGDRVDYADSVKG

SEQ ID NO: 76 - protein, 17 aa, Artificial Sequence
  synthetic peptide
TISESGGSTYYADSVKG

SEQ ID NO: 77 - protein, 17 aa, Artificial Sequence
  synthetic peptide
AFSQNGGRTDYADSVKG

SEQ ID NO: 78 - protein, 17 aa, Artificial Sequence
  synthetic peptide
TITRIGGMPYYADFVKG

SEQ ID NO: 79 - protein, 16 aa, Artificial Sequence
  synthetic peptide
GITSDDNTDYADSVKG

SEQ ID NO: 80 - protein, 16 aa, Artificial Sequence
  synthetic peptide
GITTGGVTDYADSVKG

SEQ ID NO: 81 - protein, 16 aa, Artificial Sequence
  synthetic peptide
GITKGGFADYADSVKG

SEQ ID NO: 82 - protein, 17 aa, Artificial Sequence
  synthetic peptide
GITSTGNHRNVADSVKG

SEQ ID NO: 83 - protein, 16 aa, Artificial Sequence
  synthetic peptide
AITSDGTTAYADSVKG

SEQ ID NO: 84 - protein, 17 aa, Artificial Sequence
  synthetic peptide
SIIPGSGDTSYASSVRG

SEQ ID NO: 85 - protein, 16 aa, Artificial Sequence
  synthetic peptide
GITTGGITDYTDSVKG

SEQ ID NO: 86 - protein, 16 aa, Artificial Sequence
  synthetic peptide
GITRGGDVNYADSVKG

SEQ ID NO: 87 - protein, 16 aa, Artificial Sequence
  synthetic peptide
GITSDGFTNYADSVKG

SEQ ID NO: 88 - protein, 16 aa, Artificial Sequence
  synthetic peptide
GITKGGFADYADSVKG

SEQ ID NO: 89 - protein, 16 aa, Artificial Sequence
  synthetic peptide
GITSDDKTDYADSVKD

SEQ ID NO: 90 - protein, 19 aa, Artificial Sequence
  synthetic peptide
AILWGRSGVYTTYADSVKG

SEQ ID NO: 91 - protein, 16 aa, Artificial Sequence
  synthetic peptide
GMHSGGSANYADSVKG

SEQ ID NO: 92 - protein, 16 aa, Artificial Sequence
  synthetic peptide
GITGNGLTDYADSVKG

SEQ ID NO: 93 - protein, 12 aa, Artificial Sequence
  synthetic peptide
GEYSSTAATYYY

SEQ ID NO: 94 - protein, 7 aa, Artificial Sequence
  synthetic peptide
WPEARGD

SEQ ID NO: 95 - protein, 8 aa, Artificial Sequence
  synthetic peptide
LGWPDFGS

SEQ ID NO: 96 - protein, 6 aa, Artificial Sequence
  synthetic peptide
LGFPQY

SEQ ID NO: 97 - protein, 19 aa, Artificial Sequence
  synthetic peptide
APGYMTTDSQYYLGNKYQY

SEQ ID NO: 98 - protein, 15 aa, Artificial Sequence
  synthetic peptide
GQLSSSTYYGDEYVY

SEQ ID NO: 99 - protein, 16 aa, Artificial Sequence
  synthetic peptide
SRIFSFSYADGRGYAY

SEQ ID NO: 100 - protein, 19 aa, Artificial Sequence
  synthetic peptide
RPPAYFSNAVYSKVDEYRY

SEQ ID NO: 101 - protein, 15 aa, Artificial Sequence
  synthetic peptide
DLRLRLGVTPREYDY

SEQ ID NO: 102 - protein, 7 aa, Artificial Sequence
  synthetic peptide
LPDSRLG

SEQ ID NO: 103 - protein, 7 aa, Artificial Sequence
  synthetic peptide
LGAAAYD

SEQ ID NO: 104 - protein, 6 aa, Artificial Sequence
  synthetic peptide
LPTHSD

SEQ ID NO: 105 - protein, 7 aa, Artificial Sequence
  synthetic peptide
FPDQVFN

SEQ ID NO: 106 - protein, 7 aa, Artificial Sequence
  synthetic peptide
WPQARGD

SEQ ID NO: 107 - protein, 12 aa, Artificial Sequence
  synthetic peptide
GTGGIYSSDYPY

SEQ ID NO: 108 - protein, 6 aa, Artificial Sequence
  synthetic peptide
LGMANY

SEQ ID NO: 109 - protein, 6 aa, Artificial Sequence
  synthetic peptide
LGFPSD

SEQ ID NO: 110 - protein, 6 aa, Artificial Sequence
  synthetic peptide
FPTRHY

SEQ ID NO: 111 - protein, 6 aa, Artificial Sequence
  synthetic peptide
LPTHSD

SEQ ID NO: 112 - protein, 8 aa, Artificial Sequence
  synthetic peptide
LGWPRDDY

SEQ ID NO: 113 - protein, 12 aa, Artificial Sequence
  synthetic peptide
GLGTMRANEYDY

SEQ ID NO: 114 - protein, 9 aa, Artificial Sequence
  synthetic peptide
GLGFDGHDY

SEQ ID NO: 115 - protein, 6 aa, Artificial Sequence
  synthetic peptide
LLNHAE

SEQ ID NO: 116 - protein, 232 aa, Artificial Sequence
  synthetic polypeptide
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 115 aa, Artificial Sequence
  synthetic polypeptide
EVQLVESGGGLVQPGGSLRLSCAASGITFSRSVMGWFRQAPGKLRELVSGITSDDNTDYADSVKGRFTISRDNSKNTLYLQMNSLRAEDTAVYYCARLPDSRLGWGQGTLVTVSS

SEQ ID NO: 118 - protein, 115 aa, Artificial Sequence
  synthetic polypeptide
EVQLVESGGGLVQPGGSLRLSCAASGITFSRSVMGWFRQAPGKLRELVAGITSDDNTDYADSVKGRFTISRDNSKNTLYLQMNSLRAEDTAVYYCARLPDSRLGWGQGTLVTVSS

SEQ ID NO: 119 - protein, 115 aa, Artificial Sequence
  synthetic polypeptide
EVQLVESGGGLVQPGGSLRLSCAASGITFSRSVMGWFRQAPGKLRELVAGITSDDNTDYADSVKGRFTISRDNSKNTLYLQMNSLRAEDTAVYYCAILPDSRLGWGQGTLVTVSS

SEQ ID NO: 120 - protein, 115 aa, Artificial Sequence
  synthetic polypeptide
EVQLVESGGGLVQPGGSLRLSCAASGITFSRSVMGWFRQAPGKLRELVAGITSDDNTDYADSVKGRFTISRDNSKNTLYLQMNSLRAEDTAVYYCNILPDSRLGWGQGTLVTVSS

SEQ ID NO: 121 - protein, 115 aa, Artificial Sequence
  synthetic polypeptide
EVQLVESGGGLVQPGGSLRLSCAVSGITFSRSVMGWFRQAPGKLRELVAGITSDDNTDYADSVKGRFTISRDNSKNTLYLQMNSLRAEDTAVYYCNILPDSRLGWGQGTLVTVSS

SEQ ID NO: 122 - protein, 114 aa, Artificial Sequence
  synthetic polypeptide
EVQLVESGGGLVQPGGSLRLSCAASGIRFSIANMGWYRQAPGKLHEMVSGITSDGFTNYADSVKGRFTISRDNSKNTLYLQMNSLRAEDTAVYYCAKFPTRHYWGQGTLVTVSS

SEQ ID NO: 123 - protein, 114 aa, Artificial Sequence
  synthetic polypeptide
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCAKFPTRHYWGQGTLVTVSS

SEQ ID NO: 124 - protein, 114 aa, Artificial Sequence
  synthetic polypeptide
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCASFPTRHYWGQGTLVTVSS

SEQ ID NO: 125 - protein, 114 aa, Artificial Sequence
  synthetic polypeptide
EVQLVESGGGLVQPGGSLRLSCAASGIRFSIANMGWYRQAPGKLHEMVAGITSDGFTNYADSVKGRFTISRDNSKNTLYLQMNSLRAEDTAVYYCDSFPTRHYWGQGTLVTVSS

SEQ ID NO: 126 - protein, 114 aa, Artificial Sequence
  synthetic polypeptide
EVQLVESGGGLVQPGGSLRLSCAASGSIFSPNGIGWYRQAPGKLRELVAGITGNGLTDYAESVKGRFTISRDSAKNTVYLQMNSLKTEDTAVYYCNLLLNHAEWGQGTQVTVSS

SEQ ID NO: 127 - protein, 114 aa, Artificial Sequence
  synthetic polypeptide
EVQLVESGGGLVQPGGSLRLSCAASGSIFSPNGIGWVRQAPGKGLEWVSGITGNGLTDYADSVKGRFTISRDNSKNTLYLQMNSLRAEDTAVYYCAKLLNHAEWGQGTLVTVSS

SEQ ID NO: 128 - protein, 114 aa, Artificial Sequence
  synthetic polypeptide
EVQLVESGGGLVQPGGSLRLSCAASGSIFSPNGIGWVRQAPGKGLEWVSGITGNGLTDYAESVKGRFTISRDNSKNTVYLQMNSLRAEDTAVYYCALLLNHAEWGQGTLVTVSS

SEQ ID NO: 129 - protein, 114 aa, Artificial Sequence
  synthetic polypeptide
EVQLVESGGGLVQPGGSLRLSCAASGSIFSPNGIGWYRQAPGKGRELVSGITGNGLTDYAESVKGRFTISRDNSKNTLYLQMNSLRAEDTAVYYCAKLLNHAEWGQGTLVTVSS

SEQ ID NO: 130 - protein, 114 aa, Artificial Sequence
  synthetic polypeptide
EVQLVESGGGLVQPGGSLRLSCAASGSIFSPNGIGWYRQAPGKGRELVSGITGNGLTDYAESVKGRFTISRDNSKNTVYLQMNSLRAEDTAVYYCALLLNHAEWGQGTLVTVSS

SEQ ID NO: 131 - protein, 114 aa, Artificial Sequence
  synthetic polypeptide
EVQLVESGGGLVQPGGSLRLSCAASGSIFSPNGIGWYRQAPGKLRELVAGITGNGLTDYAESVKGRFTISRDNSKNTVYLQMNSLRAEDTAVYYCNLLLNHAEWGQGTLVTVSS

SEQ ID NO: 132 - protein, 114 aa, Artificial Sequence
  synthetic polypeptide
EVQLVESGGGLVQPGGSLRLSCAASGSIFSPNAIGWVRQAPGKGLEWVSGITGNGLTDYAESVKGRFTISRDNSKNTVYLQMNSLRAEDTAVYYCALLLNHAEWGQGTLVTVSS

SEQ ID NO: 133 - protein, 114 aa, Artificial Sequence
  synthetic polypeptide
EVQLVESGGGLVQPGGSLRLSCAASGSIFSPNAIGWYRQAPGKGRELVSGITGNGLTDYAESVKGRFTISRDNSKNTLYLQMNSLRAEDTAVYYCAKLLNHAEWGQGTLVTVSS

SEQ ID NO: 134 - protein, 114 aa, Artificial Sequence
  synthetic polypeptide
EVQLVESGGGLVQPGGSLRLSCAASGSIFSPNAIGWYRQAPGKGRELVSGITGNGLTDYAESVKGRFTISRDNSKNTVYLQMNSLRAEDTAVYYCALLLNHAEWGQGTLVTVSS

SEQ ID NO: 135 - protein, 114 aa, Artificial Sequence
  synthetic polypeptide
EVQLVESGGGLVQPGGSLRLSCAASGSIFSPNAIGWYRQAPGKLRELVAGITGNGLTDYAESVKGRFTISRDNSKNTVYLQMNSLRAEDTAVYYCNLLLNHAEWGQGTLVTVSS

SEQ ID NO: 136 - protein, 114 aa, Artificial Sequence
  synthetic polypeptide
EVQLVESGGGLVQPGGSLRLSCAASGSIFSPQGIGWVRQAPGKGLEWVSGITGNGLTDYAESVKGRFTISRDNSKNTVYLQMNSLRAEDTAVYYCALLLNHAEWGQGTLVTVSS

SEQ ID NO: 137 - protein, 114 aa, Artificial Sequence
  synthetic polypeptide
EVQLVESGGGLVQPGGSLRLSCAASGSIFSPQGIGWYRQAPGKGRELVSGITGNGLTDYAESVKGRFTISRDNSKNTLYLQMNSLRAEDTAVYYCAKLLNHAEWGQGTLVTVSS

SEQ ID NO: 138 - protein, 114 aa, Artificial Sequence
  synthetic polypeptide
EVQLVESGGGLVQPGGSLRLSCAASGSIFSPQGIGWYRQAPGKGRELVSGITGNGLTDYAESVKGRFTISRDNSKNTVYLQMNSLRAEDTAVYYCALLLNHAEWGQGTLVTVSS

SEQ ID NO: 139 - protein, 114 aa, Artificial Sequence
  synthetic polypeptide
EVQLVESGGGLVQPGGSLRLSCAASGSIFSPQGIGWYRQAPGKLRELVAGITGNGLTDYAESVKGRFTISRDNSKNTVYLQMNSLRAEDTAVYYCNLLLNHAEWGQGTLVTVSS

SEQ ID NO: 140 - protein, 16 aa, Artificial Sequence
  synthetic peptide
GITGNGLTDYAESVKG

SEQ ID NO: 141 - protein, 5 aa, Artificial Sequence
  synthetic peptide
PNAIG

SEQ ID NO: 142 - protein, 5 aa, Artificial Sequence
  synthetic peptide
PQGIG

(19) United States
(12) Patent Application Publication
(10) Pub. No.: US 2024/0150460 A1
(43) Pub. Date: May 9, 2024

(54) ANTI-NKP30 ANTIBODY AND APPLICATION THEREOF

(71) Applicant: Shenghe (China) Biopharmaceutical Co., Ltd., Nanjing (CN)

(72) Inventors: Jinhua ZHOU, Nanjing (CN); Cailin ZHU, Nanjing (CN); Chongbing WU, Nanjing (CN); Xiaoling JIANG, Nanjing (CN); Liusong YIN, Nanjing (CN)

(21) Appl. No.: 18/279,014

(22) PCT Filed: Feb. 25, 2022
(86) PCT No.: PCT/CN2022/077787

§ 371 Date: Aug. 25, 2023

(30) Foreign Application Priority Data
Feb. 26, 2021 (CN) 202110213137.5
Dec. 2, 2021 (WO) PCT/CN2021/134907

(51) Int. Cl. C07K 16/28; C07K 14/475; C07K 14/495; C07K 14/535; C07K 14/54; C07K 14/555; C07K 16/30; C07K 16/32

(52) U.S. Cl. CPC C07K 16/2803; C07K 14/475; C07K 14/495; C07K 14/535; C07K 14/54; C07K 14/5421; C07K 14/5428; C07K 14/5443; C07K 14/555; C07K 16/28; C07K 16/2827; C07K 16/283; C07K 16/2866; C07K 16/2878; C07K 16/2887; C07K 16/2896; C07K 16/303; C07K 16/3069; C07K 16/32; C07K 2317/31; C07K 2317/569

(57) ABSTRACT

The present invention discloses an anti-NKp30 single domain antibody capable of activating the release of cytokines from NK cells or γδT cells, and a nucleic acid encoding the anti-NKp30 single domain antibody. The present invention also discloses a multifunctional fusion protein comprising the anti-NKp30 single domain antibody and a composition thereof, and the use in drugs for the treatment, prevention or diagnosis of diseases.

### TECHNICAL FIELD

The present invention belongs to the field of tumor immunotherapy and molecular immunology, and specifically relates to an anti-NKp30 single domain antibody and use thereof.

### BACKGROUND

Tumor is currently a major disease that endangers human health worldwide. The abnormal function of the body's immune system is very closely related to the occurrence and progression of tumors, and T-cell immune checkpoint therapies such as CTLA-4 and PD-1 have significantly improved the prognosis of patients with a variety of metastatic and refractory cancers, however, they are only effective in a small number of patients, with an efficiency rate of about 20%, and face the problem of drug resistance.

NK cells are recognized by the medical community as the first line of defense. Compared to other anti-cancer immune cells, NK cells have a stronger and more effective killing effect on tumor and virus-infected cells. Their activation is not dependent on tumor cell surface antigens, nor do they require the immune system's antigen recognition response, such as T cells, to determine the ‘attack’ target. NK cells are present in the blood vessels throughout the body to perform an immune surveillance function. They can detect and rapidly activate immune defense and immune stability functions, killing cells with pathological or cancerous changes. NK cells have a killing effect on target cells that can be observed within 1 hour in vitro and 4 hours in vivo. The major receptors that activate NK cells in humans include CD16, NKG2D and natural cytotoxic receptors (NCRs), which include NKp30, NKp44 and NKp46.

Currently, the activation of NK cells is mainly through the binding of CD16 by the Fc region of the antibody, but the affinity between Fc and CD16 is low. Scientists have also developed CD16 agonists, which can more effectively cause the activation of NK cells and exert anti-tumor effects, but the absence of CD16 or the polymorphism of CD16 limits the use of CD16 agonists.

γδT cells are immune cells that both kill cancer cells and tumor stem cells and recognize cancer antigens. They are more potent at killing cancer cells, but not as potent as NK cells. At the same time, γδT cells are mainly distributed on skin and mucosal tissues, making them excellent for treating cancers of the mucosa, such as cancers of the digestive tract, respiratory tract and reproductive system. There are no reports of biological agonists for γδT cells.

NKp30 (Natural cytotoxicity triggering receptor 3) is encoded by the NCR3 gene and is a member of the natural cytotoxicity triggering receptor (NCRs) family, an activating receptor on the cell surface. NKp30 is expressed on all resting and activated NK cells, multiple effector NKT cells, γδT cells, and MAIT cells (mucosa-associated constant T lymphocytes) and activates tumor-killing cells such as NK cells and γδT cells. Importantly, NKp30 can activate NK cells without CD16A binding and the killing effect after activation is stronger than anti-CD16A, and the anti-NKp30 antibody has a synergistic amplification effect with the anti-CD16A antibody.

Camelids or alpacas can produce a heavy chain antibody that naturally lacks the light chain. Their molecule contains only one variable heavy chain region (VHH) and two conventional CH2 and CH3 regions, but has full antigen binding capabilities and does not aggregate as easily as the artificially modified single chain antibody fragments (scFv). Due to their special structural properties, heavy chain single domain antibodies combine the advantages of both conventional antibodies and small molecule drugs, overcoming the drawbacks of conventional antibodies such as long development cycles, low stability and harsh preservation conditions, and represent a new generation of antibody therapeutic development.

Although the single domain antibody is much smaller in size than the conventional monoclonal antibody with two heavy and two light chains, it can bind antigens with similar affinity and specificity to monoclonal antibodies (mAbs). When a single domain antibody is used as a building block, it can be fused into the IgG Fc domain to produce IgG-like antibodies, both bivalent and polyvalent. Development of bispecific antibody targeting tumor-associated antigens and NKp30 can bridge tumor cells and NK cells. Activating only NK cells in the tumor microenvironment helps to reduce side effects and avoid reduced sensitivity to abnormal cells that lose MHC molecules due to high activation of systemic NK cells.

The present invention uses NKp30 as the target for immunotherapy and develops new anti-NKp30 single domain antibodies for the development of bifunctional antibodies, multifunctional antibodies or multifunctional fusion proteins.

### SUMMARY

The present invention provides an anti-NKp30 single domain antibody, wherein the antibody can activate the release of cytokines from NK cells or γδT cells.

In alternative embodiments, the cytokine is a lymphokine, preferably IL2, IL3, IL4, IL5, IL6, IL9, IL10, IFN-γ or TNF-α, and more preferably IFN-γ, TNF-α or IL2.

In alternative embodiments, the anti-NKp30 single domain antibody comprises immunoglobulin single variable domain, wherein the immunoglobulin single variable domain comprises complementary determining regions CDR1, CDR2 and CDR3, wherein,
(a) CDR1, which is selected from any amino acid sequence of SEQ ID NOs: 47-69, 141 and 142, or a sequence having at least 80%, 85%, 90%, 91%, 92%, 93%, 94%, 95%, 96%, 97%, 98%, 99% or more identity to any amino acid sequence of SEQ ID NOs: 47-69, 141 and 142, or an amino acid sequence having one or more (preferably 2 or 3) conserved amino acid mutations (preferably substitutions, insertions or deletions) compared to any amino acid sequence of SEQ ID NOs: 47-69, 141 and 142;
(b) CDR2, which is selected from any amino acid sequence of SEQ ID NOs: 70-92 and 140, or a sequence having at least 80%, 85%, 90%, 91%, 92%, 93%, 94%, 95%, 96%, 97%, 98%, 99% or more identity to any amino acid sequence of SEQ ID NOs: 70-92 and 140, or an amino acid sequence having one or more (preferably 2 or 3) conserved amino acid mutations (preferably substitutions, insertions or deletions) compared to any amino acid sequence of SEQ ID NOs: 70-92 and 140;
(c) CDR3, which is selected from any amino acid sequence of SEQ ID NOs: 93-115, or a sequence having at least 80%, 85%, 90%, 91%, 92%, 93%, 94%, 95%, 96%, 97%, 98%, 99% or more identity to any amino acid sequence of SEQ ID NOs: 93-115, or an amino acid sequence having one or more (preferably 2 or 3) conserved amino acid mutations (preferably substitutions, insertions or deletions) compared to any amino acid sequence of SEQ ID NOs: 93-115.

In alternative embodiments, the anti-NKp30 single variable domain comprises a CDR1, CDR2 and CDR3 selected from:
(1) CDR1 shown by SEQ ID NO: 47, CDR2 shown by SEQ ID NO: 70, and CDR3 shown by SEQ ID NO: 93;
(2) CDR1 shown by SEQ ID NO: 48, CDR2 shown by SEQ ID NO: 71, and CDR3 shown by SEQ ID NO: 94;
(3) CDR1 shown by SEQ ID NO: 49, CDR2 shown by SEQ ID NO: 72, and CDR3 shown by SEQ ID NO: 95;
(4) CDR1 shown by SEQ ID NO: 50, CDR2 shown by SEQ ID NO: 73, and CDR3 shown by SEQ ID NO: 96;
(5) CDR1 shown by SEQ ID NO: 51, CDR2 shown by SEQ ID NO: 74, and CDR3 shown by SEQ ID NO: 97;
(6) CDR1 shown by SEQ ID NO: 52, CDR2 shown by SEQ ID NO: 75, and CDR3 shown by SEQ ID NO: 98;
(7) CDR1 shown by SEQ ID NO: 53, CDR2 shown by SEQ ID NO: 76, and CDR3 shown by SEQ ID NO: 99;
(8) CDR1 shown by SEQ ID NO: 54, CDR2 shown by SEQ ID NO: 77, and CDR3 shown by SEQ ID NO: 100;
(9) CDR1 shown by SEQ ID NO: 55, CDR2 shown by SEQ ID NO: 78, and CDR3 shown by SEQ ID NO: 101;
(10) CDR1 shown by SEQ ID NO: 56, CDR2 shown by SEQ ID NO: 79, and CDR3 shown by SEQ ID NO: 102;
(11) CDR1 shown by SEQ ID NO: 57, CDR2 shown by SEQ ID NO: 80, and CDR3 shown by SEQ ID NO: 103;
(12) CDR1 shown by SEQ ID NO: 58, CDR2 shown by SEQ ID NO: 81, and CDR3 shown by SEQ ID NO: 104;
(13) CDR1 shown by SEQ ID NO: 59, CDR2 shown by SEQ ID NO: 82, and CDR3 shown by SEQ ID NO: 105;
(14) CDR1 shown by SEQ ID NO: 60, CDR2 shown by SEQ ID NO: 83, and CDR3 shown by SEQ ID NO: 106;
(15) CDR1 shown by SEQ ID NO: 61, CDR2 shown by SEQ ID NO: 84, and CDR3 shown by SEQ ID NO: 107;
(16) CDR1 shown by SEQ ID NO: 62, CDR2 shown by SEQ ID NO: 85, and CDR3 shown by SEQ ID NO: 108;
(17) CDR1 shown by SEQ ID NO: 63, CDR2 shown by SEQ ID NO: 86, and CDR3 shown by SEQ ID NO: 109;
(18) CDR1 shown by SEQ ID NO: 64, CDR2 shown by SEQ ID NO: 87, and CDR3 shown by SEQ ID NO: 110;
(19) CDR1 shown by SEQ ID NO: 65, CDR2 shown by SEQ ID NO: 88, and CDR3 shown by SEQ ID NO: 111;
(20) CDR1 shown by SEQ ID NO: 66, CDR2 shown by SEQ ID NO: 89, and CDR3 shown by SEQ ID NO: 112;
(21) CDR1 shown by SEQ ID NO: 67, CDR2 shown by SEQ ID NO: 90, and CDR3 shown by SEQ ID NO: 113;
(22) CDR1 shown by SEQ ID NO: 68, CDR2 shown by SEQ ID NO: 91, and CDR3 shown by SEQ ID NO: 114;
(23) CDR1 shown by SEQ ID NO: 69, CDR2 shown by SEQ ID NO: 92, and CDR3 shown by SEQ ID NO: 115;
(24) CDR1 shown by SEQ ID NO: 69, CDR2 shown by SEQ ID NO: 140, and CDR3 shown by SEQ ID NO: 115;
(25) CDR1 shown by SEQ ID NO: 141, CDR2 shown by SEQ ID NO: 140, and CDR3 shown by SEQ ID NO: 115, and/or
(26) CDR1 shown by SEQ ID NO: 142, CDR2 shown by SEQ ID NO: 140, and CDR3 shown by SEQ ID NO: 115.

In alternative embodiments, the immunoglobulin single variable domain is VHH.

In alternative embodiments, the VHH comprises a sequence having at least 80%, 85%, 90%, 91%, 92%, 93%, 94%, 95%, 96%, 97%, 98%, 99% or more identity to any amino acid sequence in SEQ ID NOs: 1-23 and 117-139.

In alternative embodiments, the VHH is selected from any amino acid sequence in SEQ ID NOs: 1-23.

In alternative embodiments, the VHH is selected from any amino acid sequence in SEQ ID NOs: 117-139.

In alternative embodiments, the antibody binds NKp30 at a KD of 20.1 nM or less.

In alternative embodiments, the antibody comprises an immunoglobulin Fc region, and the immunoglobulin Fc region is selected from IgG1, IgG2, IgG3 and/or IgG4.

In alternative embodiments, the amino acid sequence of the immunoglobulin Fc region is as shown by SEQ ID NO: 116.

The present invention also provides a nucleic acid molecule encoding an anti-NKp30 single domain antibody as described in any one of the above.

The invention also provides an expression vector comprising nucleic acid molecules as described above operably linked to an expression regulatory element.

The invention also provides a recombinant cell comprising the nucleic acid molecules as described above or transformed with the expression vectors as described above and capable of expressing the anti-NKp30 single domain antibody.

The present invention also provides a multifunctional fusion protein comprising the anti-NKp30 single domain antibody as described in any one of the above.

In alternative embodiments, the multifunctional fusion protein further comprises one or more secondary antibody or antigen-binding portions thereof that binds specifically to other antigens.

In alternative embodiments, the antigen binding the secondary antibody or antigen-binding portion thereof is selected from a tumor associated antigen (TAA) or an immune checkpoint.

In alternative embodiments, the tumor associated antigen (TAA) is selected from BCMA, CD38, HER2, PSMA, Claudin18.2, GPC3, CD19, CD20 (MS4A1), CD22, CD24, CD30, CD33, CD38, CD40, CD123, CD133, CD138, CDK4, CEA, AFP, ALK or B7H3.

In alternative embodiments, the secondary antibody or antigen-binding portion thereof is a NK cell agonist.

In alternative embodiments, the antigen binding the secondary antibody or antigen-binding portion thereof is selected from NKP30, NKP46, CD16, NKP44, CD244, CD226, NKG2E, NKG2D, NKG2C or KIR.

In alternative embodiments, the multifunctional fusion protein thereof further comprises a cytokine.

In alternative embodiments, the cytokine is selected from IL8, IL10, IL15, IL18, TGF, VEGF, IFNγ, IFNα or GM-CSF.

The present invention also provides the use of the anti-NKp30 single domain antibody and the multifunctional fusion protein as described in any one of the above in the preparation of drugs for the treatment and/or prevention and/or diagnosis of diseases.

In alternative embodiments, the use is achieved by one or more of tumor immunotherapy, cell therapy and gene therapy.

The present invention also provides the use of the anti-NKp30 single domain antibodies and multifunctional fusion proteins as described in any one of the above in drugs for the treatment of cancer.

In alternative embodiments, the cancer is lung cancer, liver cancer, melanoma, malignant glioma, head and neck cancer, colorectal cancer, gastric cancer, prostate cancer, ovarian cancer, bladder cancer, pancreatic cancer, gastric cancer, colon cancer, cervical cancer or related tumors.

The present invention also provides a pharmaceutical composition, wherein the pharmaceutical composition comprises the anti-NKp30 single domain antibody as described in any one of the above and an acceptable carrier, diluent or excipient.

The present invention also provides a pharmaceutical composition, wherein the pharmaceutical composition comprises the multifunctional fusion protein as described in any one of the above and an acceptable carrier, diluent or excipient.

The present invention provides an anti-NKp30 single domain antibody that can specifically bind to NKp30, activate the NK immune response, and promote the release of cytokines, such as IFN-γ and TNF-α, from NK cells; the above function is close to or exceeds the level of the current NKp30 monoclonal antibody.

To assist in the understanding of the invention described herein, the following explanations of abbreviations and definitions of terms are provided.

The following abbreviations are used herein:
IgG: Immunoglobulin G;
ELISA: Enzyme-linked immunosorbent assay;
FACS: Fluorescence-activated cell sorting.

The term “domain” (of a peptide or protein) refers to a folded protein structure that can maintain its tertiary structure independently of the rest of the protein. In general, domains are responsible for the individual functional properties of a protein and in many cases can be added, removed, or transferred to other proteins without losing the rest of the protein and/or the function of the domain.

The term “tumor-associated antigen” or “TAA” refers to a molecule (typically a protein, carbohydrate, lipid, or some combination of them) that is expressed completely or as fragments on the surface of cancerous cells and that can be used to prioritize the targeting of pharmacological agents to cancerous cells. Non-limiting examples of “tumor-associated antigens” include, for example, BCMA, CD38, HER2, PSMA, Claudin 18.2, GPC3, CD19, CD20 (MS4A1), CD22, CD24, CD30, CD33, CD38, CD40, CD123, CD133, CD138, CDK4, CEA, AFP, ALK or B7H3.

The terms “antibody” or “immunoglobulin” used herein interchangeably, whether referring to heavy chain antibodies or regular 4-chain antibodies, are used as general terms to include full-length antibodies, their individual chains, and all of their parts, domains or fragments (including, but not limited to, antigen-binding domains or fragments, such as the VHH domain or VH/VL domain, respectively). Furthermore, the term “sequence” as used herein (for example in terms such as “immunoglobulin sequence”, “antibody sequence”, “single variable domain sequence”, “VHH sequence” or “protein sequence”) should generally be understood to include both the relevant amino acid sequence and the nucleic acid or nucleotide sequence encoding said sequence, unless a more restricted interpretation is required herein.

The term “immunoglobulin single variable domain” refers to an immunoglobulin variable domain that is capable of specifically binding an antigenic epitope without pairing with other immunoglobulin variable domains. An example of an immunoglobulin single variable domain within the meaning of the present invention is a “domain antibody”, for example, an immunoglobulin single variable domain is a “VHH domain” (or simply “VHH”) of the Camelidae family as defined below.

“VHH domain”, also known as heavy chain single domain antibody, single domain antibody, VHH, VHH domain, VHH antibody fragment and VHH antibody, is a variable domain of antigen-binding immunoglobulins called “heavy chain antibodies” (i.e. “antibodies lacking light chains”) (Hamers-Casterman C, Atarhouch T, Muyldermans S, Robinson G, Hamers C, Songa EB, BendahmanN, Hamers R.: “Naturally occurring antibodies devoid of light chains”; Nature 363, 446-448 (1993)). The term “VHH domain” is used to distinguish the variable domain from the heavy chain variable domain present in conventional 4-chain antibodies (which is referred to herein as the “VH domain”) and the light chain variable domain present in conventional 4-chain antibodies (which is referred to herein as the “VL domain”). The VHH domain binds epitopes specifically without the need for other antigen-binding domains (this contrasts with the VH or VL domains in conventional 4-chain antibodies, where the epitopes are recognized by the VL domain together with the VH domain). The VHH domain is a small, stable and efficient antigen recognition unit formed by a single immunoglobulin domain.

In the context of the present invention, the terms “single domain antibody”, “heavy chain single domain antibody”, “VHH domain”, “VHH”, “VHH domain”, “VHH antibody fragment”, “VHH antibody”, “nanobody” and “Nanobody” are used interchangeably.

The term “immunoglobulin variable domain” refers to an antibody domain consisting essentially of four “framework regions” referred to in the art and hereinafter as “framework region 1” or “FR1”, “framework region 2” or “FR2”, “framework region 3” or “FR3”, and “framework region 4” or “FR4”, respectively, wherein the framework regions are separated by three “complementary determining regions” or “CDRs” referred to in the art and hereinafter as “complementary determining region 1” or “CDR1”, “complementary determining region 2” or “CDR2”, and “complementary determining region 3” or “CDR3”, respectively. The general structure or sequence of the variable domain of an immunoglobulin can therefore be expressed as follows: FR1-CDR1-FR2-CDR2-FR3-CDR3-FR4. The variable domain of immunoglobulin gives the antibody its specificity to the antigen due to the presence of the antigen-binding site.

The term “specificity” refers to the number of different types of antigens or epitopes to which a particular antigen-binding molecule or antigen-binding protein (such as the immunoglobulin single variable domain of the present invention) can bind. The specificity of an antigen-binding protein can be determined based on its affinity and/or avidity. The affinity expressed by the dissociation equilibrium constant (KD) of the antigen to the antigen-binding protein is a measure of the strength of the binding between the epitope and the antigen-binding site on the antigen-binding protein: the smaller the KD value, the stronger the binding strength between the epitope and the antigen-binding protein (alternatively, the affinity can also be expressed as the association constant (KA), which is 1/KD). As will be appreciated by those skilled in the art, depending on the specific antigen of interest, the affinity can be determined in a known manner. The avidity is a measure of the strength of binding between an antigen-binding protein (such as an immunoglobulin, an antibody, an immunoglobulin single variable domain or a polypeptide containing it) and the associated antigen. The avidity is related to both the avidity between the antigen-binding sites on its antigen-binding protein and the number of relevant binding sites present on the antigen binding protein.

The term “polypeptide” refers to a chain of amino acids of any length, independent of modifications (such as phosphorylation or glycosylation). The term polypeptide includes proteins and fragments thereof. Polypeptides can be “exogenous”, meaning that they are “heterologous”, i.e. foreign to the host cell being utilized, such as human polypeptides produced by bacterial cells. Herein, polypeptides are disclosed as sequences of amino acid residues. Those sequences are written from left to right in the direction of the amino terminus to the carboxyl terminus. According to standard nomenclature, amino acid residue sequences are named with a three-letter or single-letter code as follows: alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y) and valine (Val, V).

The “percent (%) amino acid sequence identity” with respect to the reference polypeptide sequence is defined as the percentage of amino acid residues in the candidate sequence that are identical to those in the reference polypeptide sequence after the sequence is aligned and a notch is introduced if necessary to obtain the maximum percentage sequence identity. Comparisons for the purpose of determining percentage amino acid sequence identity may be made in a variety of ways within the technical scope of the art, for example using publicly available computer software such as BLAST, BLAST-2, Clustal W, Megalign (DNASTAR) software or the FASTA package.

The term “host cell” refers to a cell that has been or is capable of being transformed with a nucleic acid sequence and thereby expressing the selected target gene. The term includes the progeny of a parental cell, whether or not the progeny is morphologically or genetically identical to the original parental cell, provided that the selected target gene is present in the progeny. Commonly used host cells include bacteria, yeast, mammalian cells, etc.

The term “transfection” refers to the uptake of foreign or exogenous DNA into a cell, and the technique can be used to introduce one or more exogenous DNA fractions into a suitable host cell. Cells can be induced by physicochemical methods (for example, by calcium chloride treatment) so that they are in a physiological state that is best suited to take up and accommodate foreign DNA, namely the “competent state”.

The term “vector” refers to a nucleic acid molecule capable of proliferating another nucleic acid to which it is attached. The term includes vectors that act as self-replicating nucleic acid structures and are incorporated into the genome of the host cell receiving their introduction. Some vectors can direct the expression of nucleic acids to which they are operationally linked. Such vectors are referred to herein as “expression vectors”.

### BRIEF DESCRIPTION OF THE DRAWINGS

FIGS. 1a-1b show the FACS results of the Chimeric anti-NKp30 single domain antibody binding to a stable expression cell line.

FIGS. 2a-2c show the experimental results of the Chimeric anti-NKp30 single domain antibody stimulating NK cells to activate and release cytokines.

FIGS. 3a-3h show the FACS results of the Humanized anti-NKp30 antibody and/or CDR-modified anti-NKp30 antibody binding to a stable expression cell line.

FIG. 4 shows the experimental results of the Humanized anti-NKp30 antibody stimulating NK cells to activate and release cytokines.

### DETAILED DESCRIPTION

The present invention is further described below in connection with the accompanying drawings and specific embodiments, the protection of which is not limited to the following embodiments. It should also be understood that the terms used in embodiments of the present invention are intended to describe specific embodiments and are not intended to limit the scope of protection of the present invention. Without departing from the spirit and scope of the inventive concept, variations and advantages that can be envisaged by those skilled in the art are included in the present invention and the scope of protection of the invention by the appended claims and any equivalents thereof. In the specification and claims of the invention, the singular forms “a”, “one” and “this” include the plural forms unless the context expressly states otherwise. The processes, conditions, reagents, experimental methods, etc. for carrying out the invention are, except where specifically mentioned, of general knowledge and common knowledge to those skilled in the art, and the invention is not specifically limited in the present invention.

The invention is illustrated below by means of more specific embodiments.

### Example 1 Animal Immunization

The recombinant human NKp30, Fc tag protein (ACRO, Cat: NC3-H5259) was prepared as the immunogen. The total amount of antigen per immunization was kept between 1-2 mg, and the volume was less than 2 mL. The antigen and adjuvant were emulsified in 1: 1 to form a homogeneous mixture and stored at 4° C. After recording the number of the camel's ears, the immunization experiment was started, and each time the camels were injected into the left and right sides of the neck lymph nodes at 2 points on each side, and 0.4 mL of mixed antigen was injected into each point. After immunization, the camels were observed for half an hour to confirm that they were in good condition and had no uncomfortable symptoms. Immunization was performed on day 0, day 21, day 42 and day 63, respectively. 10 mL of blood was collected from the camel neck vein on day 28, 50 mL blood was collected on day 49 and day 70, respectively, and part of blood was taken out for serum titer detection each time. Immunization was given every 2 weeks for a total of 7 immunizations. Blood was collected at an interval of 5-7 days after the 6th and 7th immunization, with 25-30 mL of blood collected each time and divided into 3 collection vessels. Before the 4th, 5th and 6th immunization, blood was collected for immune evaluation. 5 mL of blood was collected from camel neck vein each time. The blood was centrifuged at 25° C. and 400× g for 30 minutes on the same day to separate and preserve the upper serum. Then the lymphocytes were separated, that is, 3 mL of cell separation fluid was first added to the 15 mL centrifuge tube, and then 3 mL of blood was slowly added. The blood was added slowly and carefully to prevent mixing of the blood and the separation solution. Then, the centrifuge was precooled to room temperature and centrifuged at 400 g for 30 minutes. The separation of the blood in the centrifuge tube was observed. The middle cotton upper layer immune cells were carefully absorbed into a new 15 mL centrifuge tube with a 200 μL pipette. The upper serum was stored in a new centrifuge tube at −80° C. 10 mL of PBS buffer placed at room temperature was added to each tube and centrifuged at 25° C. and 400 g for 20 min to remove the supernatant. 5 mL of PBS buffer placed at room temperature was added to each tube and centrifuged at 25° C. and 400 g for 20 min. The cell count was calculated using a blood cell counting plate. The supernatant was removed, and the lymphocytes isolated by RNAiso Plus were dissolved according to the number of cells to obtain 107/mL lysate, and stored at −80° C.

### Example 2 Constructing Phage Library

PBMC was isolated with lymphocyte separation fluid at the second and third blood samples. Total RNA from PBMC was extracted and reverse transcribed by PrimeScript™ II 1st Strand cDNA Synthesis Kit (Takara, item No. 6210A) with a total of 5 μg RNA. The cDNA stock solution was mixed in equal proportions and diluted 5 times, 5.0 μL was added for the first round of amplification, and the amplified product was gelled for recovery. The recovered product was used as the template for the second round of amplification, and the amplified product was gelled for recovery as the target fragment. The vector and the target fragment were enzymatically cut with SfiI, and the target fragment was recovered after overnight digestion at 50° C. The link molar ratio was Vector: VHH=1:3. A total of 10 times of electric transformation were performed. Immediately after the shock, 1 mL of 2YT medium (preheated at 37° C.) was added to the shock cup for resuscitation, the shock products were sucked out and the shock cup was washed with 2YT medium. A total of 100 mL resuscitation products were obtained, and resuscitation was performed at 37° C. and 180 rpm for 45 min. Gradient dilution of 100 μL was taken to 10-3 and 10-4 to determine the number of converters in the library, and coated on a 90 mm plate, and the rest was centrifuged. Then, 8 mL of 2YT was added to resuspend, and coated on eight 200 mm plates. On the second day, the number of converters in the library was measured and the capacity of the library was calculated.

The bacterial library was inserted into 2×300 mL 2YT+A+G (Amp: 100 μg/mL, Glu: 1%) medium until its initial OD600=0.1-0.2, and incubated at 37° C. and 230 rpm until OD600=0.8 or above. The auxiliary phage M13KO7 was added according to the OD600 value (auxiliary phage: bacteria=20: 1). After adding M13KO7, it was mixed well and left to stand at 37° C. for 30 min. It was shaken slowly at 37° C. and 180 rpm for 30 min. It was centrifuged at 5000 rpm for 10 min, and then the supernatant was discarded fully. The precipitate was resuspended with an equal volume of 2YT+A+K (Amp: 100 μg/mL, Kan: 50 μg/mL) medium at 30° C. and 220 rpm overnight. Overnight cultures were centrifuged at 4° C. and 10,000 rpm for 20 min, and then the supernatant was collected, and the precipitate was discarded. The centrifuge cartridge was replaced, and then centrifuged at 4° C. and 10000 rpm for 20 min, and the supernatant was collected. PEG8000/NaCl was added by ⅕ of the supernatant volume, and then it was mixed well and precipitated for more than 2 hours in an ice bath. It was centrifuged at 4° C. and 10,000 rpm for 20 min, and then the supernatant was discarded and centrifuged empty once to fully remove the supernatant. 1 mL of 1×PBS was used to suspend the precipitate, and PEG8000/NaCl was added by ⅕ of the supernatant volume to precipitate again for 1 h. It was centrifuged at 4° C. and 12000 rpm for 10 min, and then supernatant was discarded and centrifuged again to fully remove the supernatant. According to the amount of precipitate, 1×PBS was added to resuspend the precipitate. 100% glycerol was added until the final concentration was 50%, mixed well and distributed into 1.5 mL EP tubes and stored at −80° C. 10 μL of library phage was taken and diluted with 2YT gradient. 10 μL was taken from 10-8 and 10-9 tubes and added to 90 μL of TG1 bacteriophage solution, and mixed gently. It wasstand at 37° C. for 15 min, Amp resistant plates were coated separately and incubated overnight. The next day, the titer of cloned metal phage library on the titer plate was calculated.

### Example 3 Phage Library Screening for Positive Antibody

The target molecule NKp30his was diluted with carbonate buffer at pH 9.6 to a final concentration of 5 μg/mL, and added to the enzyme-labelled wells at 100 μL/well. Each target molecule was coated with 8 wells (It was coated with 4 wells in the second round of screening, and it was coated with 2 wells in each of the third and fourth rounds of screening), and the wells were coated at 4° C. overnight. The coating solution was discarded, and washed 3 times with PBS. 300 μL of 3% BSA-PBS blocking solution was added to each well, and blocked at 37° C. for 1 h. The wells were washed 3 times with PBS, and then 100 μL of phage library was added, and they were incubated at 37° C. for 1 h. The unbound phage was aspirated, and then the wells were washed with PBST for 6 times, and washed with PBS for 2 times. 100 μL of Gly-HCl eluate was added and incubated at 37° C. for 8 min to elute specifically bound phage. The eluate was transferred to a 1.5 mL sterile centrifuge tube and quickly neutralized with 10 μL of Tris-HCl neutralization buffer. 10 μL was taken for gradient dilution, and then the titer was measured, and the panning recovery was calculated. The remaining eluate was mixed, amplified and purified for the next round of affinity panning.

The panning eluate was mixed with 5 mL of E. coli TG1 culture in the pre-logarithmic growth phase, and allowed to stand for 30 min at 37° C., and incubated at 220 r/min with shaking for 30 min. It was centrifuged at 1000 g for 15 min, and then the supernatant was removed, and coated with 500 μL of 2×YT resuspension onto a 200 mm 2×YT-GA plate. The bacteria were scraped with 10 mL of 2×YT liquid medium, and 500 μL of suspension was added into 50 mL of 2×YT liquid medium, and then it was shaken at 37° C. for 30 min. M13K07 auxiliary phage was added at the ratio of cell: phage=1:20, and allowed to stand for 30 min at 37° C., and shaken at 220 r/min for 30 min. The cultures were distributed in centrifuge tubes, and centrifuged at 25° C. and 5000 r/min for 10 min. The cell precipitates were resuspended in 50 mL of 2×YT-AK liquid medium, and incubated overnight at 30° C. and 230 r/min with shaking. Overnight cultures were centrifuged at 4° C. and 10000 r/min for 20 min, and the supernatant was transferred to a new centrifuge tube. PEG/NaCl was added by ⅕ of the supernatant volume, and then it was mixed well and placed at 4° C. for more than 2 h. It was centrifuged at 4° C. and 10000 r/min for 20 min, and the supernatant was removed. The precipitate was resuspended in 1 mL PBS, and then PEG/NaCl was added by ⅕ of the supernatant volume. It was mixed well and placed at 4° C. for more than 1 h. It was centrifuged at 4° C. and 12000 r/min for 2 min. The supernatant was removed, and then the precipitate was suspended in 200 μL PBS, and the amplification product was obtained. The titer was determined for the next round of panning or analysis.

From the plate of panning eluent titer, 96 clones (numbered 1-96) were randomly selected from the plate of the second round of titer assay using a sterilized toothpick. 96 clones (numbered 97-192) were randomly selected from the first round of titer plate, and then inoculated in 1 mL of 2×YT-A, and incubated at 37° C. and 230 r/min with shaking for 8 h. 200 μL of the above culture was taken, and M13K07 phage was added at the ratio of cell: phage=1:20. It was allowed to stand for 15 min at 37° C., and incubated at 220 r/min with shaking for 45 min. 800 μL of 2×YT-AK was added, and incubated at 30° C. with vigorous shaking overnight. The next day, it was centrifuged at 12,000 rpm for 2 min, and then supernatant was taken and used for monoclonal ELISA identification.

The target molecule NKp30 antigen was diluted with carbonate buffer at pH 9.6 to a final concentration of 2 μg/mL, and added to the enzyme-labelled wells per 100 μL/well, and coated at 4° C. overnight. The coating solution was discarded and washed 3 times with PBST. 300 μL of 5% skimmed milk was added to each well, and the wells were blocked at 37° C. for 1 h. The wells were washed 3 times with PBST, and 50 μL of phage culture supernatant and 50 μL of 5% skimmed milk were added to each well, and the wells were incubated at 37° C. for 1 h. The wells were washed 5 times with PBST, and anti-M13 antibody labeled with horseradish peroxidase was added (diluted with PBS at 1: 10,000) at 100 μL/well and treated at 37° C. for 1 h. The plate was washed 6 times with PBST. TMB color developing fluid was added at 100 L/well for color development at 37° C. for 7 min. Termination solution was added at 50 μL/well to terminate the reaction, and the optical density was measured at 450 nm.

### Example 4 Sequencing of Antibody Gene Sequence

The sequences obtained from phage library screening were subjected to antibody gene sequencing. 23 antibodies were selected, and their amino acid/nucleotide sequences are as follows:
(1) the amino acid sequences of CDR1, CDR2 and CDR3 of the variable domain of sequence 1 are SEQ ID NO: 47, SEQ ID NO: 70 and SEQ ID NO: 93, respectively;
   the amino acid sequence of the variable domain of sequence 1 is SEQ ID NO: 1; and
   the nucleotide sequence of the variable domain of sequence 1 is SEQ ID NO: 24.
(2) the amino acid sequences of CDR1, CDR2 and CDR3 of the variable domain of sequence 2 are SEQ ID NO: 48, SEQ ID NO: 71 and SEQ ID NO: 94, respectively;
   the amino acid sequence of the variable domain of sequence 2 is SEQ ID NO: 2; and
   the nucleotide sequence of the variable domain of sequence 2 is SEQ ID NO: 25.
(3) the amino acid sequences of CDR1, CDR2 and CDR3 of the variable domain of sequence 3 are SEQ ID NO: 49, SEQ ID NO: 72 and SEQ ID NO: 95, respectively;
   the amino acid sequence of the variable domain of sequence 3 is SEQ ID NO: 3; and
   the nucleotide sequence of the variable domain of sequence 3 is SEQ ID NO: 26.
(4) the amino acid sequences of CDR1, CDR2 and CDR3 of the variable domain of sequence 4 are SEQ ID NO: 50, SEQ ID NO: 73 and SEQ ID NO: 96, respectively;
   the amino acid sequence of the variable domain of sequence 4 is SEQ ID NO: 4; and
   the nucleotide sequence of the variable domain of sequence 4 is SEQ ID NO: 27.
(5) the amino acid sequences of CDR1, CDR2 and CDR3 of the variable domain of sequence 5 are SEQ ID NO: 51, SEQ ID NO: 74 and SEQ ID NO: 97, respectively;
   the amino acid sequence of the variable domain of sequence 5 is SEQ ID NO: 5; and
   the nucleotide sequence of the variable domain of sequence 5 is SEQ ID NO: 28.
(6) the amino acid sequences of CDR1, CDR2 and CDR3 of the variable domain of sequence 6 are SEQ ID NO: 52, SEQ ID NO: 75 and SEQ ID NO: 98, respectively;
   the amino acid sequence of the variable domain of sequence 6 is SEQ ID NO: 6; and
   the nucleotide sequence of the variable domain of sequence 6 is SEQ ID NO: 29.
(7) the amino acid sequences of CDR1, CDR2 and CDR3 of the variable domain of sequence 7 are SEQ ID NO: 53, SEQ ID NO: 76 and SEQ ID NO: 99, respectively;
   the amino acid sequence of the variable domain of sequence 7 is SEQ ID NO: 7; and
   the nucleotide sequence of the variable domain of sequence 7 is SEQ ID NO: 30.
(8) the amino acid sequences of CDR1, CDR2 and CDR3 of the variable domain of sequence 8 are SEQ ID NO: 54, SEQ ID NO: 77 and SEQ ID NO: 100, respectively;
   the amino acid sequence of the variable domain of sequence 8 is SEQ ID NO: 8; and
   the nucleotide sequence of the variable domain of sequence 8 is SEQ ID NO: 31.
(9) the amino acid sequences of CDR1, CDR2 and CDR3 of the variable domain of sequence 9 are SEQ ID NO: 55, SEQ ID NO: 78 and SEQ ID NO: 101, respectively;
   the amino acid sequence of the variable domain of sequence 9 is SEQ ID NO: 9; and
   the nucleotide sequence of the variable domain of sequence 9 is SEQ ID NO: 32.
(10) the amino acid sequences of CDR1, CDR2 and CDR3 of the variable domain of sequence 10 are SEQ ID NO: 56, SEQ ID NO: 79 and SEQ ID NO: 102, respectively;
   the amino acid sequence of the variable domain of sequence 10 is SEQ ID NO: 10; and
   the nucleotide sequence of the variable domain of sequence 10 is SEQ ID NO: 33.
(11) the amino acid sequences of CDR1, CDR2 and CDR3 of the variable domain of sequence 11 are SEQ ID NO: 57, SEQ ID NO: 80 and SEQ ID NO: 103, respectively;
   the amino acid sequence of the variable domain of sequence 11 is SEQ ID NO: 11; and
   the nucleotide sequence of the variable domain of sequence 11 is SEQ ID NO: 34.
(12) the amino acid sequences of CDR1, CDR2 and CDR3 of the variable domain of sequence 12 are SEQ ID NO: 58, SEQ ID NO: 81 and SEQ ID NO: 104, respectively;
   the amino acid sequence of the variable domain of sequence 12 is SEQ ID NO: 12; and
   the nucleotide sequence of the variable domain of sequence 12 is SEQ ID NO: 35.
(13) the amino acid sequences of CDR1, CDR2 and CDR3 of the variable domain of sequence 13 are SEQ ID NO: 59, SEQ ID NO: 82 and SEQ ID NO: 105, respectively;
   the amino acid sequence of the variable domain of sequence 13 is SEQ ID NO: 13; and
   the nucleotide sequence of the variable domain of sequence 13 is SEQ ID NO: 36.
(14) the amino acid sequences of CDR1, CDR2 and CDR3 of the variable domain of sequence 14 are SEQ ID NO: 60, SEQ ID NO: 83 and SEQ ID NO: 106, respectively;
   the amino acid sequence of the variable domain of sequence 14 is SEQ ID NO: 14; and
   the nucleotide sequence of the variable domain of sequence 14 is SEQ ID NO: 37.
(15) the amino acid sequences of CDR1, CDR2 and CDR3 of the variable domain of sequence 15 are SEQ ID NO: 61, SEQ ID NO: 84 and SEQ ID NO: 107, respectively;
   the amino acid sequence of the variable domain of sequence 15 is SEQ ID NO: 15; and
   the nucleotide sequence of the variable domain of sequence 15 is SEQ ID NO: 38.
(16) the amino acid sequences of CDR1, CDR2 and CDR3 of the variable domain of sequence 16 are SEQ ID NO: 62, SEQ ID NO: 85 and SEQ ID NO: 108, respectively;
   the amino acid sequence of the variable domain of sequence 16 is SEQ ID NO: 16; and
   the nucleotide sequence of the variable domain of sequence 16 is SEQ ID NO: 39.
(17) the amino acid sequences of CDR1, CDR2 and CDR3 of the variable domain of sequence 17 are SEQ ID NO: 63, SEQ ID NO: 86 and SEQ ID NO: 109, respectively;
   the amino acid sequence of the variable domain of sequence 17 is SEQ ID NO: 17; and
   the nucleotide sequence of the variable domain of sequence 17 is SEQ ID NO: 40.
(18) the amino acid sequences of CDR1, CDR2 and CDR3 of the variable domain of sequence 18 are SEQ ID NO: 64, SEQ ID NO: 87 and SEQ ID NO: 110, respectively;
   the amino acid sequence of the variable domain of sequence 18 is SEQ ID NO: 18; and
   the nucleotide sequence of the variable domain of sequence 18 is SEQ ID NO: 41.
(19) the amino acid sequences of CDR1, CDR2 and CDR3 of the variable domain of sequence 19 are SEQ ID NO: 65, SEQ ID NO: 88 and SEQ ID NO: 111, respectively;
   the amino acid sequence of the variable domain of sequence 19 is SEQ ID NO: 19; and
   the nucleotide sequence of the variable domain of sequence 19 is SEQ ID NO: 42.
(20) the amino acid sequences of CDR1, CDR2 and CDR3 of the variable domain of sequence 20 are SEQ ID NO: 66, SEQ ID NO: 89 and SEQ ID NO: 112, respectively;
   the amino acid sequence of the variable domain of sequence 20 is SEQ ID NO: 20; and
   the nucleotide sequence of the variable domain of sequence 20 is SEQ ID NO: 43.
(21) the amino acid sequences of CDR1, CDR2 and CDR3 of the variable domain of sequence 21 are SEQ ID NO: 67, SEQ ID NO: 90 and SEQ ID NO: 113, respectively;
   the amino acid sequence of the variable domain of sequence 21 is SEQ ID NO: 21; and
   the nucleotide sequence of the variable domain of sequence 21 is SEQ ID NO: 44.
(22) the amino acid sequences of CDR1, CDR2 and CDR3 of the variable domain of sequence 22 are SEQ ID NO: 68, SEQ ID NO: 91 and SEQ ID NO: 114, respectively;
the amino acid sequence of the variable domain of sequence 22 is SEQ ID NO: 22; and
the nucleotide sequence of the variable domain of sequence 22 is SEQ ID NO: 45.
(23) the amino acid sequences of CDR1, CDR2 and CDR3 of the variable domain of sequence 23 are SEQ ID NO: 69, SEQ ID NO: 92 and SEQ ID NO: 115, respectively;
   the amino acid sequence of the variable domain of sequence 23 is SEQ ID NO: 23; and
   the nucleotide sequence of the variable domain of sequence 23 is SEQ ID NO: 46.

### Example 5 Construction and Expression of Chimeric Antibodies

The sequences obtained from phage library screening were sequenced for antibody genes, and then the sequenced antibody fragments were gene synthesized and constructed into a human IgG framework. Then, the antibody fragment was inserted into PCDNA3.1 vector using molecular cloning technology to construct mammalian cell expression plasmid, which was introduced into the host cell line CHO cells using liposome transfection. The fermentation supernatant was obtained by cell fed-batch, and the supernatant of fermentation liquid was purified by affinity chromatography, ion exchange chromatography and so on. Finally, the constructed chimeric antibodies were purified and obtained: antibody 1, antibody 2, antibody 3, antibody 4, antibody 5, antibody 6, antibody 7, antibody 8, antibody 9, antibody 10, antibody 11, antibody 12, antibody 13, antibody 14, antibody 15, antibody 16, antibody 17, antibody 18, antibody 19, antibody 20, antibody 21, antibody 22 and antibody 23. The amino acid sequences of the CDR and variable domains of antibody 1 to antibody 23 correspond to the amino acid sequences of the CDR and variable domains of sequence 1 to sequence 23 in Example 4, respectively.

The constant region of antibody 1 to antibody 23 all have the same amino acid sequence as shown by SEQ ID NO: 116.

### Example 6 Affinity Verification of Anti-NKP30 Antibodies and NKp30

Equipment: OCTET Red96e (Fortebio).

Sensor: AHC.

(1) Experimental Setup:

Sensor preparation: the AHC sensor was soaked in 0.02% PBST (0.02% Tween 20, pH 7.4, 1*PBS) as buffer for 600 s prior to use to remove the sucrose covering the sensor surface.

The sample plate and sensor position were set according to the actual addition position of the sample.

The steps to be performed, time and speed were set. The experimental temperature was set to 30° C. and the shaking speed was set to 1000 rpm.

(2) Immobilization and Capture:

After equilibration of the AHC sensor with 0.02% PBST (0.02% Tween 20, pH 7.4, 1*PBS) as buffer for 60 s, the NKp30 antibody in the sample plate was immobilized for 300 s and second equilibration was performed for 180 s. 100 nm human NKp30-his protein (KACTUS; Cat: NKP-HM430) was bound with NKp30 antibody for 300 s and then dissociated for 600 s. After dissociation, 10 mM glycine (pH 2.0) was used as regeneration buffer to regenerate for 30 s.

(3) Regeneration:

The sensor was regenerated with 10 mM glycine (pH 2.0).

(4) Data Analysis:

Subtract the result plot for reference channel H1 from the test result plot. The experimental data fit the 1:1 binding model. The molecular weight of 35 kDa was used to calculate the molar concentration of human NKp30 protein. The results are shown in Table 1.

As shown by Table 1, except for antibody 5 with a KD of 1.04×10⁻⁷ M and antibody 21 with a KD of 2.01×10⁻⁸ M, the KD values of other antibodies were at the nanomolar concentration level or even smaller, indicating that the NKp30 single domain antibodies have a high affinity with NKp30.

TABLE 1

| Sample No. | K_{D} (M) |
|---|---|
| Antibody 1 | 1.46 × 10⁻⁹ |
| Antibody 2 | 5.97 × 10⁻¹⁰ |
| Antibody 3 | 2.07 × 10⁻⁹ |
| Antibody 4 | 4.97 × 10⁻⁹ |
| Antibody 5 | 1.04 × 10⁻⁷ |
| Antibody 6 | 2.88 × 10⁻⁹ |
| Antibody 7 | 9.64 × 10⁻⁹ |
| Antibody 8 | 9.11 × 10⁻⁹ |
| Antibody 9 | 5.56 × 10⁻⁹ |
| Antibody 10 | 2.01 × 10⁻¹⁰ |
| Antibody 11 | 3.06 × 10⁻⁹ |
| Antibody 12 | 4.91 × 10⁻¹⁰ |
| Antibody 13 | 4.33 × 10⁻¹⁰ |
| Antibody 14 | 1.35 × 10⁻⁹ |
| Antibody 15 | 3.47 × 10⁻¹⁰ |
| Antibody 16 | 9.97 × 10⁻¹⁰ |
| Antibody 17 | 1.26 × 10⁻⁹ |
| Antibody 18 | 3.02 × 10⁻¹⁰ |
| Antibody 19 | 1.81 × 10⁻⁹ |
| Antibody 20 | 1.25 × 10⁻⁹ |
| Antibody 21 | 2.01 × 10⁻⁸ |
| Antibody 22 | 2.31 × 10⁻⁹ |
| Antibody 23 | 2.34 × 10⁻⁹ |

### Example 7 Test for Binding Capacity Between Antibody and Cell

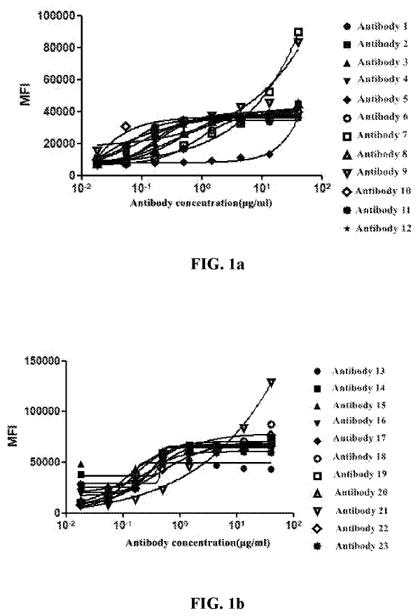

The antibody to be tested was taken, and the initial concentration was 40 μg/mL, and then diluted at 1: 3 for 8 gradients dilutions. The NK cells in the incubator were taken out, and the cell suspension was transferred to a 15 mL centrifuge tube, and then centrifuged, and resuspended in PBS for counting. Blank control (Blank), negative control (NC), experimental group and irrelevant antibody group were set. The cell suspension was spread in a 96-well plate according to approximately 3×105 cells/well. After centrifugation (1000 rpm, 5 min), the plate was washed with PBS and centrifuged again, which was repeated twice to remove the medium residue. The supernatant was discarded, and 100 μL of primary antibody solution and irrelevant antibody solution were added to experimental group and irrelevant antibody group, respectively. The cells were resuspended, and incubated at room temperature for 1 h. Blank group and NC group were incubated with an equal amount of PBS. After 1 h, centrifugation was performed, and PBS was added and washed twice. After the supernatant was discarded, 100 μL of fluorescent secondary antibody dilution (goat anti-human Fc-FITC Abcam, Cat: ab97224) was added to each sample group, except Blank group to which 100 μL of PBS was added. The samples were incubated at room temperature and protected from light for 0.5 h. Centrifugation was performed, and PBS was added and washed twice. After the supernatant was discarded, 120 L of PBS buffer was added to resuspend and flow cytometry was performed sequentially to measure the average fluorescence intensity. The results were shown in FIG. 1a and FIG. 1b.

The analysis shows that the anti-NKp30 antibodies all have relatively good binding activity.

### Example 8 Experiment of the Release of Cytokines by Antibody-Stimulated NK Cell Activation

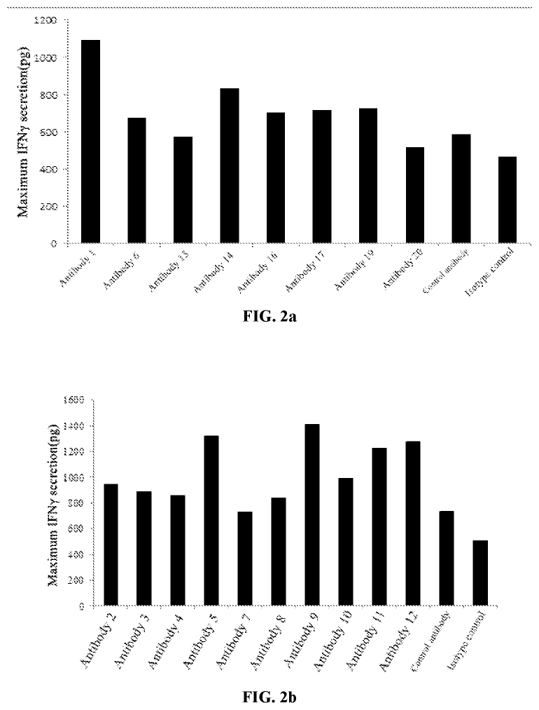

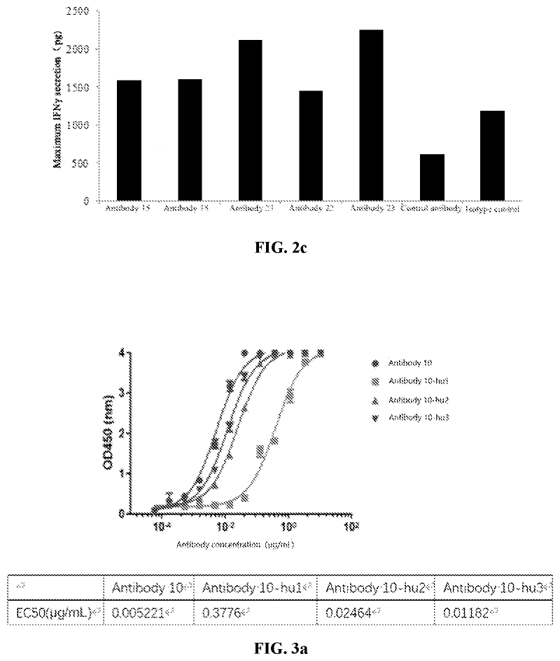

The 96-well plate was taken out, and the antibody to be tested, positive control antibody (selected from anti-CD337 (NKp30) antibody of Biolegend) and isotype control antibody which has initial concentration of 150 nM were diluted at 1: 3 for 7 gradients dilutions. Two parallel wells were set, and then dissolved in PBS buffer, and placed in the 96-well plate. The plate was incubated at 4° C. in a refrigerator for about 16 h overnight, and then taken out for subsequent operations. The 96-well plate was taken out, and then the antibody incubation solution was discarded, and washed with PBS for 2 times. The NK cells were taken out and cell counting was performed. The cell number was set to 4×104 cells/well. It was resuspended with medium containing IL-2 (STEMCELL, 78036) at a final concentration of 400 U. It was added to the 96-well plate at 200 μL/well where the antibody incubation was completed, and blank control and negative control wells were set. The treated 96-well plates were placed in a 37° C. and CO2 constant temperature incubator for about 24 h, and then the supernatants were extracted, and the kit (Biolegend, Cat: 430104) was used to measure the maximum IFN-γ secretion of the centrifuged supernatants. The results are shown in FIGS. 2a-2c.

The analysis shows that cytokine release from NK cells stimulated by the anti-NKp30 antibodies are significantly better than that of the control antibody and isotype antibody.

### Example 9 Antibody Humanization

The camelid antibody sequences obtained in Example 5 were compared to the human Germline sequences using the IgBLAST tool, and the results showed that the framing regions 1-3 of the variable region of antibody 10 contained 15 camelid antibody sites (VHH genes), and the framing regions 1-3 of the variable region of antibody 18 contained 14 camelid antibody sites (VHH genes). IGHV3 category was selected as the design template to design the humanized sequence and mutate the sequence into the humanized sequence. Five humanized antibodies with three identical CDRS were obtained from antibody 10: antibody 10−hu1, antibody 10−hu2, antibody 10−hu3, antibody 10−hu4 and antibody 10−hu5, and the amino acid sequences of CDR1, CDR2 and CDR3 of the variable domains were SEQ ID NO: 56, SEQ ID NO: 79 and SEQ ID NO: 102, respectively. Four humanized antibodies with three identical CDRS were obtained from antibody 18: antibody 18−hu1, antibody 18−hu2, antibody 18−hu3 and antibody 18−hu4, and the amino acid sequences of CDR1, CDR2 and CDR3 of the variable domains were SEQ ID NO: 64, SEQ ID NO: 87 and SEQ ID NO: 110, respectively.

The amino acid sequences of the variable domains of the humanized antibodies were shown in Table 2.

TABLE 2

| Humanized antibodies | Variable region amino acid sequences |
|---|---|
| Antibody 10-hu1 | SEQ ID NO: 117 |
| Antibody 10-hu2 | SEQ ID NO: 118 |
| Antibody 10-hu3 | SEQ ID NO: 119 |
| Antibody 10-hu4 | SEQ ID NO: 120 |
| Antibody 10-hu5 | SEQ ID NO: 121 |
| Antibody 18-hu1 | SEQ ID NO: 122 |
| Antibody 18-hu2 | SEQ ID NO: 123 |
| Antibody 18-hu3 | SEQ ID NO: 124 |
| Antibody 18-hu4 | SEQ ID NO: 125 |

The constant region of 9 humanized antibodies in Table 2 all have the same amino acid sequence as shown by SEQ ID NO: 116.

### Example 10 Humanization and CDR Modification of Antibodies

The CDR2 of antibody 23 in Example 5 was modified by post-translational modification, i.e., the amino acid sequence of CDR2 was modified from GITGNGLTDYADSVKG to GITGNGLTDYAESVKG to obtain 1 chimeric antibody: antibody 23-p.

Antibody 23 in Example 5 was humanized according to the method of Example 9, and the CDR was modified with post-translational modifications to give 13 humanized antibodies.

The 14 antibody sequences obtained were shown in Table 3

TABLE 3

| | variable structure | | | |
|---|---|---|---|---|
| Antibody Name | domain | CDR1 | CDR2 | CDR3 |
| Antibody23-p | SEQ ID NO: 126 | SEQ ID NO: 69 | SEQ ID NO: 140 | SEQ ID NO: 115 |
| Antibody23-hu41 | SEQ ID NO: 127 | SEQ ID NO: 69 | SEQ ID NO: 92 | SEQ ID NO: 115 |
| Antibody23-hu42 | SEQ ID NO: 128 | SEQ ID NO: 69 | SEQ ID NO: 140 | SEQ ID NO: 115 |
| Antibody23-hu43 | SEQ ID NO: 129 | SEQ ID NO: 69 | SEQ ID NO: 140 | SEQ ID NO: 115 |
| Antibody23-hu44 | SEQ ID NO: 130 | SEQ ID NO: 69 | SEQ ID NO: 140 | SEQ ID NO: 115 |
| Antibody23-hu45 | SEQ ID NO: 131 | SEQ ID NO: 69 | SEQ ID NO: 140 | SEQ ID NO: 115 |
| Antibody23-hu46 | SEQ ID NO: 132 | SEQ ID NO: 141 | SEQ ID NO: 140 | SEQ ID NO: 115 |
| Antibody23-hu47 | SEQ ID NO: 133 | SEQ ID NO: 141 | SEQ ID NO: 140 | SEQ ID NO: 115 |
| Antibody23-hu48 | SEQ ID NO: 134 | SEQ ID NO: 141 | SEQ ID NO: 140 | SEQ ID NO: 115 |
| Antibody23-hu49 | SEQ ID NO: 135 | SEQ ID NO: 141 | SEQ ID NO: 140 | SEQ ID NO: 115 |
| Antibody23-hu50 | SEQ ID NO: 136 | SEQ ID NO: 142 | SEQ ID NO: 140 | SEQ ID NO: 115 |
| Antibody23-hu51 | SEQ ID NO: 137 | SEQ ID NO: 142 | SEQ ID NO: 140 | SEQ ID NO: 115 |
| Antibody23-hu52 | SEQ ID NO: 138 | SEQ ID NO: 142 | SEQ ID NO: 140 | SEQ ID NO: 115 |
| Antibody23-hu53 | SEQ ID NO: 139 | SEQ ID NO: 142 | SEQ ID NO: 140 | SEQ ID NO: 115 |

The constant region of 14 antibodies in Table 3 all have the same amino acid sequence as shown by SEQ ID NO: 116.

### Example 11 Test for Binding Capacity Between Humanized Antibodies and Cell

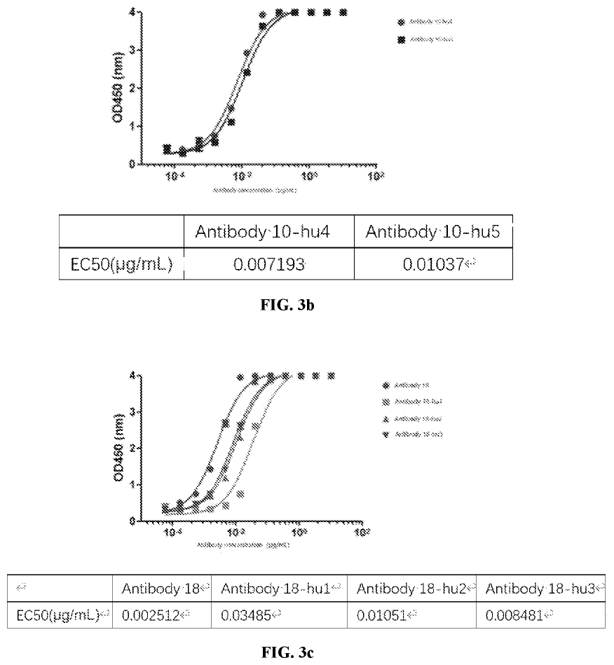

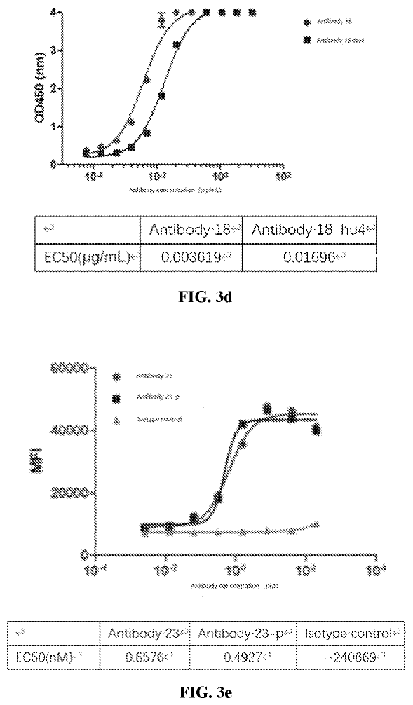

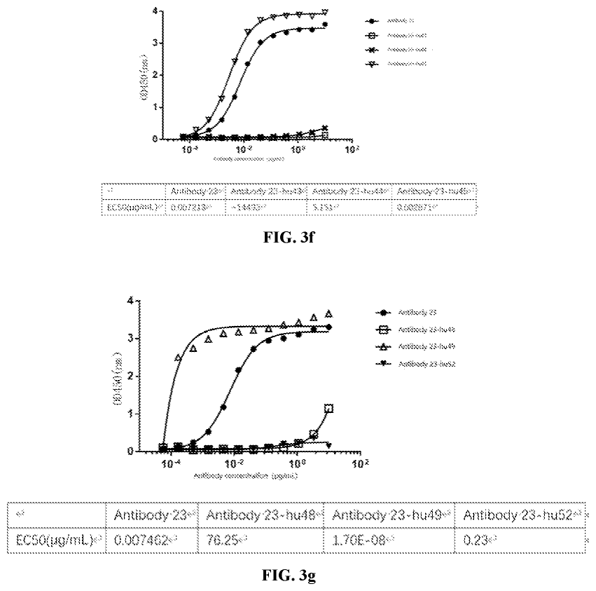

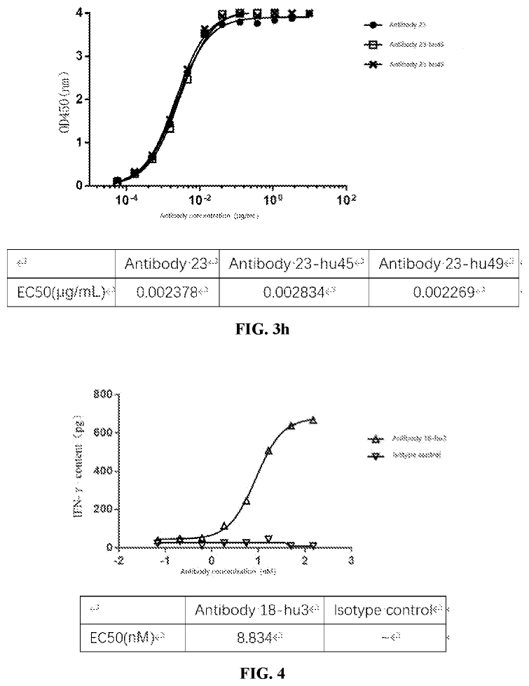

Human-NKp30-His (purchased from KACTUS, item No. NKP-HM430) was diluted to 0.2 μg/mL with the coating solution (1×PBS, pH 7.4), and was coated into 96-well enzyme-labelled plates at 100 μL/well and allowed to stand overnight at 4° C. The coating solution was poured off, the plate was washed with 1×PBST at 300 μL/well, washed with a plate washer for 4 times, and patted dry on a plate paper. The plate was blocked with 3% skimmed milk powder at 300 μL/well, and incubated at 37° C. for 1 h. The blocking solution was poured off, washed with a plate washer for 4 times, and patted dry on a plate paper. The reference product and the test product were diluted with 3% skimmed milk powder to 10 μg/mL, and the initial concentration was diluted by 3 times. A total of 11 gradients were diluted, and another blank well was set, and only diluent was added. It was incubated at 37° C. and 100 μL/well for 1 h. Liquid in the wells was discarded, and washed with a plate washer for 4 times, and patted dry on a plate paper. The goat anti-human Fc was diluted with 3% skimmed milk powder at 1: 20,000 and 100 μL/well, and incubated at 37° C. for 1 h. The plate was washed with a plate washer for 6 times, and patted dry on a plate paper. TMB color developing solution was added at 100 μL/well, and wrapped with an aluminum foil, and the color was developed at 37° C. for 8 minutes, protected from light. The termination solution 1 M HCl was added at 100 μL/well to terminate the color development reaction. It was read at 450 nm on an enzyme-labeled instrument. The results are shown in FIGS. 3a-3h.

As shown in FIGS. 3a-3b, humanized antibody 10−hu1, antibody 10−hu2, antibody 10−hu3, antibody 10−hu4 and antibody 10−hu5 have a cell binding capacity superior to or equivalent to chimeric antibody 10.

As shown in FIGS. 3c-3d, humanized antibody 18−hu1, antibody 18−hu2, antibody 18−hu3 and antibody 18−hu4 have a cell binding capacity equivalent to chimeric antibody 18.

As shown in FIGS. 3e-3h, antibody 23-p, antibody 23−hu44, antibody 23−hu45 and antibody 23−hu49 have a cell binding capacity significantly better than or equivalent to chimeric antibody 23.

### Example 12 Affinity Test for Humanized Antibodies

The affinity of the CDR-modified and humanized antibodies was tested according to the method of Example 6, and the results are shown in Table 4.

TABLE 4

| Antibody Name | Affinity K_{D} (M) |
|---|---|
| Antibody 23 | 8.44 × 10⁻¹⁰ |
| Antibody 23-p | 1.27 × 10⁻⁹ |
| Antibody 23-hu43 | 3.77 × 10⁻¹⁰ |
| Antibody 23-hu44 | 1.30 × 10⁻⁹ |
| Antibody 23-hu45 | 9.91 × 10⁻¹⁰ |
| Antibody 23-hu48 | 5.10 × 10⁻⁹ |
| Antibody 23-hu52 | 5.93 × 10⁻⁹ |

As shown in Table 4, the KD values of antibody 23, antibody 23-p, antibody 23-hu43, antibody 23−hu44, antibody 23−hu45, antibody 23−hu48, and antibody 23−hu52 are all at the nanomolar concentration level or even smaller, indicating that the NKp30 single domain antibody has a high affinity to NKp30.

### Example 13 Experiment of the Release of Cytokines by Humanized Antibody-Stimulated NK Cell Activation

According to the method of Example 8, the humanized antibody 18−hu3 was used to stimulate NK cells to activate and release cytokines, and the experimental results are shown in FIG. 4.

The experimental results show that the NK cells stimulated by antibody 18−hu3 can secrete IFN-γ with an EC50 value of 8.834 nM, while the NK cells stimulated by all concentrations of the isotype control do not produce IFN-γ, indicating that the antibody 18−hu3 can activate NK cells specifically.

The present invention is not limited in its protection to the above embodiments. Without departing from the spirit and scope of the inventive concept, variations and advantages that can be thought of by those skilled in the art are included in the present invention and are protected by the appended claims.

1. (canceled)

2. (canceled)

3. An anti-NKp30 single domain antibody comprising an immunoglobulin single variable domain, wherein the immunoglobulin single variable domain comprises complementary determining regions CDR1, CDR2 and CDR3, wherein,
(a) CDR1, which is selected from any amino acid sequence of SEQ ID NOs: 47-69, 141 and 142, or a sequence having at least 80%, 85%, 90%, 91%, 92%, 93%, 94%, 95%, 96%, 97%, 98%, 99% or more identity to any amino acid sequence of SEQ ID NOs: 47-69, 141 and 142, or an amino acid sequence having one or more (preferably 2 or 3) conserved amino acid mutations (preferably substitutions, insertions or deletions) compared to any amino acid sequence of SEQ ID NOs: 47-69, 141 and 142;
(b) CDR2, which is selected from any amino acid sequence of SEQ ID NOs: 70-92 and 140, or a sequence having at least 80%, 85%, 90%, 91%, 92%, 93%, 94%, 95%, 96%, 97%, 98%, 99% or more identity to any amino acid sequence of SEQ ID NOs: 70-92 and 140, or an amino acid sequence having one or more (preferably 2 or 3) conserved amino acid mutations (preferably substitutions, insertions or deletions) compared to any amino acid sequence of SEQ ID NOs: 70-92 and 140;
(c) CDR3, which is selected from any amino acid sequence of SEQ ID NOs: 93-115, or a sequence having at least 80%, 85%, 90%, 91%, 92%, 93%, 94%, 95%, 96%, 97%, 98%, 99% or more identity to any amino acid sequence of SEQ ID NOs: 93-115, or an amino acid sequence having one or more (preferably 2 or 3) conserved amino acid mutations (preferably substitutions, insertions or deletions) compared to any amino acid sequence of SEQ ID NOs: 93-115.

4. The anti-NKp30 single domain antibody of claim 1, wherein the single variable domain comprises CDR1, CDR2 and CDR3 selected from:
(1) CDR1 shown by SEQ ID NO: 47, CDR2 shown by SEQ ID NO: 70, and CDR3 shown by SEQ ID NO: 93;
(2) CDR1 shown by SEQ ID NO: 48, CDR2 shown by SEQ ID NO: 71, and CDR3 shown by SEQ ID NO: 94;
(3) CDR1 shown by SEQ ID NO: 49, CDR2 shown by SEQ ID NO: 72, and CDR3 shown by SEQ ID NO: 95;
(4) CDR1 shown by SEQ ID NO: 50, CDR2 shown by SEQ ID NO: 73, and CDR3 shown by SEQ ID NO: 96;
(5) CDR1 shown by SEQ ID NO: 51, CDR2 shown by SEQ ID NO: 74, and CDR3 shown by SEQ ID NO: 97;
(6) CDR1 shown by SEQ ID NO: 52, CDR2 shown by SEQ ID NO: 75, and CDR3 shown by SEQ ID NO: 98;
(7) CDR1 shown by SEQ ID NO: 53, CDR2 shown by SEQ ID NO: 76, and CDR3 shown by SEQ ID NO: 99;
(8) CDR1 shown by SEQ ID NO: 54, CDR2 shown by SEQ ID NO: 77, and CDR3 shown by SEQ ID NO: 100;
(9) CDR1 shown by SEQ ID NO: 55, CDR2 shown by SEQ ID NO: 78, and CDR3 shown by SEQ ID NO: 101;
(10) CDR1 shown by SEQ ID NO: 56, CDR2 shown by SEQ ID NO: 79, and CDR3 shown by SEQ ID NO: 102;
(11) CDR1 shown by SEQ ID NO: 57, CDR2 shown by SEQ ID NO: 80, and CDR3 shown by SEQ ID NO: 103;
(12) CDR1 shown by SEQ ID NO: 58, CDR2 shown by SEQ ID NO: 81, and CDR3 shown by SEQ ID NO: 104;
(13) CDR1 shown by SEQ ID NO: 59, CDR2 shown by SEQ ID NO: 82, and CDR3 shown by SEQ ID NO: 105;
(14) CDR1 shown by SEQ ID NO: 60, CDR2 shown by SEQ ID NO: 83, and CDR3 shown by SEQ ID NO: 106;
(15) CDR1 shown by SEQ ID NO: 61, CDR2 shown by SEQ ID NO: 84, and CDR3 shown by SEQ ID NO: 107;
(16) CDR1 shown by SEQ ID NO: 62, CDR2 shown by SEQ ID NO: 85, and CDR3 shown by SEQ ID NO: 108;
(17) CDR1 shown by SEQ ID NO: 63, CDR2 shown by SEQ ID NO: 86, and CDR3 shown by SEQ ID NO: 109;
(18) CDR1 shown by SEQ ID NO: 64, CDR2 shown by SEQ ID NO: 87, and CDR3 shown by SEQ ID NO: 110;
(19) CDR1 shown by SEQ ID NO: 65, CDR2 shown by SEQ ID NO: 88, and CDR3 shown by SEQ ID NO: 111;
(20) CDR1 shown by SEQ ID NO: 66, CDR2 shown by SEQ ID NO: 89, and CDR3 shown by SEQ ID NO: 112;
(21) CDR1 shown by SEQ ID NO: 67, CDR2 shown by SEQ ID NO: 90, and CDR3 shown by SEQ ID NO: 113;
(22) CDR1 shown by SEQ ID NO: 68, CDR2 shown by SEQ ID NO: 91, and CDR3 shown by SEQ ID NO: 114;
(23) CDR1 shown by SEQ ID NO: 69, CDR2 shown by SEQ ID NO: 92, and CDR3 shown by SEQ ID NO: 115;
(24) CDR1 shown by SEQ ID NO: 69, CDR2 shown by SEQ ID NO: 140, and CDR3 shown by SEQ ID NO: 115;
(25) CDR1 shown by SEQ ID NO: 141, CDR2 shown by SEQ ID NO: 140, and CDR3 shown by SEQ ID NO: 115; and/or
(26) CDR1 shown by SEQ ID NO: 142, CDR2 shown by SEQ ID NO: 140, and CDR3 shown by SEQ ID NO: 115.

5. The anti-NKp30 single domain antibody of claim 1, wherein the immunoglobulin single variable domain is VHH.

6. The anti-NKp30 single domain antibody of claim 3, wherein the VHH comprises a sequence having at least 80%, 85%, 90%, 91%, 92%, 93%, 94%, 95%, 96%, 97%, 98%, 99% or more identity to any amino acid sequence in SEQ ID NOs: 1-23 and 117-139.

7. The anti-NKp30 single domain antibody of claim 4, wherein the VHH is selected from any amino acid sequence in SEQ ID NOs: 1-23.

8. The anti-NKp30 single domain antibody of claim 4, wherein the VHH is selected from any amino acid sequence in SEQ ID NOs: 117-139.

9. The anti-NKp30 single domain antibody of claim 1, wherein the antibody binds NKp30 at a KD of 20.1 nM or less.

10. The anti-NKp30 single domain antibody of claim 1, wherein it further comprises an immunoglobulin Fc region, the immunoglobulin Fc region being selected from IgG1, IgG2, IgG3 and/or IgG4.

11. The anti-NKp30 single domain antibody of claim 8, wherein the amino acid sequence of the immunoglobulin Fc region being as shown by SEQ ID NO: 116.

12. A nucleic acid molecule encoding the anti-NKp30 single domain antibody of any one of claims 1 to 9.

13. (canceled)

14. (canceled)

15. A multifunctional fusion protein comprising an anti-NKp30 single domain antibody of any one of claims 1 to 9.

16. The multifunctional fusion protein of claim 11, wherein it further comprises one or more secondary antibody or antigen-binding portions thereof that bind specifically to other antigens.

17. The multifunctional fusion protein of claim 12, wherein the antigen binding the secondary antibody or antigen-binding portion thereof is selected from a tumor associated antigen (TAA) or an immune checkpoint.

18. The multifunctional fusion protein of claim 13, wherein the tumor associated antigen (TAA) is selected from BCMA, CD38, HER2, PSMA, Claudin18.2, GPC3, CD19, CD20 (MS4A1), CD22, CD24, CD30, CD33, CD38, CD40, CD123, CD133, CD138, CDK4, CEA, AFP, ALK or B7H3.

19. The multifunctional fusion protein of claim 12, wherein the secondary antibody or antigen-binding portion thereof is NK cell agonist.

20. The multifunctional fusion protein of claim 15, wherein the antigen binding the secondary antibody or antigen-binding portion thereof is selected from NKP30, NKP46, CD16, NKP44, CD244, CD226, NKG2E, NKG2D, NKG2C or KIR.

21. The multifunctional fusion protein of claim 11, wherein it further comprises a cytokine.

22. The multifunctional fusion protein of claim 1724, wherein the cytokine is selected from IL8, IL10, IL15, IL18, TGF, VEGF, IFNγ, IFNα or GM-CSF.

23. (canceled)

24. (canceled)

25. (canceled)

26. (canceled)

27. (canceled)

28. (canceled)
